SEQUENCE LISTING (30 sequences)

SEQ ID NO: 1 - protein, 12 aa, Artificial
  Synthetic
EEDTQFNYHRKK

SEQ ID NO: 2 - protein, 12 aa, Artificial
  Synthetic
EKLNKELASKNK

SEQ ID NO: 3 - protein, 12 aa, Artificial
  Synthetic
TLEENQVKKYHR

SEQ ID NO: 4 - protein, 10 aa, Artificial
  Synthetic
LKEEASKRAA

SEQ ID NO: 5 - protein, 9 aa, Artificial
  Synthetic
KADQDRLDL

SEQ ID NO: 6 - protein, 11 aa, Artificial
  Synthetic
EERKKVETEAK

SEQ ID NO: 7 - protein, 11 aa, Artificial
  Synthetic
EIEENQKRIEK

SEQ ID NO: 8 - protein, 12 aa, Artificial
  Synthetic
DRQESSRQQRKA

SEQ ID NO: 9 - protein, 12 aa, Artificial
  Synthetic
TDEKLRELKGAK

SEQ ID NO: 10 - protein, 12 aa, Artificial
  Synthetic
RYEPPHIKKALQ

SEQ ID NO: 11 - protein, 12 aa, Artificial
  Synthetic
KKRLEFENQKTR

SEQ ID NO: 12 - protein, 12 aa, Artificial
  Synthetic
QDLKNQHLAKKS

SEQ ID NO: 13 - protein, 12 aa, Artificial
  Synthetic
TKYPDANPNPNE

SEQ ID NO: 14 - protein, 11 aa, Artificial
  Synthetic
MGFLKLIEIEN

SEQ ID NO: 15 - protein, 7 aa, Artificial
  Synthetic
FKSYKGR

SEQ ID NO: 16 - protein, 12 aa, Artificial
  Synthetic
ENFKSYKGRQII

SEQ ID NO: 17 - protein, 17 aa, Artificial
  Synthetic
QRFTAIIGPNGSGKSNL

SEQ ID NO: 18 - protein, 15 aa, Artificial
  Synthetic
NGSGKSNLMDAISFV

SEQ ID NO: 19 - protein, 21 aa, Artificial
  Synthetic
DAISFVLGEKTSNLRVKTLRD

SEQ ID NO: 20 - protein, 13 aa, Artificial
  Synthetic
LIHGAPVGKPAAN

SEQ ID NO: 21 - protein, 19 aa, Artificial
  Synthetic
PAANRAFVSMVYSEEGAED

SEQ ID NO: 22 - protein, 23 aa, Artificial
  Synthetic
DRTFARVIVGGSSEYKINNKVVQ

SEQ ID NO: 23 - protein, 26 aa, Artificial
  Synthetic
HEYSEELEKLGILIKARNFLVFQGAV

SEQ ID NO: 24 - protein, 13 aa, Artificial
  Synthetic
ESIAMKNPKERTA

SEQ ID NO: 25 - protein, 21 aa, Artificial
  Synthetic
LFEEISRSGDVAQEYDKRKKE

SEQ ID NO: 26 - protein, 1233 aa, Homo sapiens
MGFLKLIEIENFKSYKGRQIIGPFQRFTAIIGPNGSGKSNLMDAISFVLGEKTSNLRVKTLRDLIHGAPVGKPAANRAFVSMVYSEEGAEDRTFARVIVGGSSEYKINNKVVQLHEYSEELEKLGILIKARNFLVFQGAVESIAMKNPKERTALFEEISRSGDVAQEYDKRKKEMVKAEEDTQFNYHRKKNIAAERKEAKQEKEEADRYQRLKDEVVRAQVQLQLFKLYHNEVEIEKLNKELASKNKEIEKDKKRMDKVEDELKEKKKELGKMMREQQQIEKEIKEKDSELNQKRPQYIKAKENTSHKIKKLEAAKKSLQNAQKHYKKRKGDMDELEKEMLSVEKARQEFEERMEEESQSQGRDLTLEENQVKKYHRLKEEASKRAATLAQELEKFNRDQKADQDRLDLEERKKVETEAKIKQKLREIEENQKRIEKLEEYITTSKQSLEEQKKLEGELTEEVEMAKRRIDEINKELNQVMEQLGDARIDRQESSRQQRKAEIMESIKRLYPGSVYGRLIDLCQPTQKKYQIAVTKVLGKNMDAIIVDSEKTGRDCIQYIKEQRGEPETFLPLDYLEVKPTDEKLRELKGAKLVIDVIRYEPPHIKKALQYACGNALVCDNVEDARRIAFGGHQRHKTVALDGTLFQKSGVISGGASDLKAKARRWDEKAVDKLKEKKERLTEELKEQMKAKRKEAELRQVQSQAHGLQMRLKYSQSDLEQTKTRHLALNLQEKSKLESELANFGPRINDIKRIIQSREREMKDLKEKMNQVEDEVFEEFCREIGVRNIREFEEEKVKRQNEIAKKRLEFENQKTRLGIQLDFEKNQLKEDQDKVHMWEQTVKKDENEIEKLKKEEQRHMKIIDETMAQLQDLKNQHLAKKSEVNDKNHEMEEIRKKLGGANKEMTHLQKEVTAIETKLEQKRSDRHNLLQACKMQDIKLPLSKGTMDDISQEEGSSQGEDSVSGSQRISSIYAREALIEIDYGDLCEDLKDAQAEEEIKQEMNTLQQKLNEQQSVLQRIAAPNMKAMEKLESVRDKFQETSDEFEAARKRAKKAKQAFEQIKKERFDRFNACFESVATNIDEIYKALSRNSSAQAFLGPENPEEPYLDGINYNCVAPGKRFRPMDNLSGGEKTVAALALLFAIHSYKPAPFFVLDEIDAALDNTNIGKVANYIKEQSTCNFQAIVISLKEEFYTKAESLIGVYPEQGDCVISKVLTFDLTKYPDANPNPNEQ

SEQ ID NO: 27 - protein, 804 aa, Homo sapiens
MGFLKLIEIENFKSYKGRQIIGPFQRFTAIIGPNGSGKSNLMDAISFVLGEKTSNLRVKTLRDLIHGAPVGKPAANRAFVSMVYSEEGAEDRTFARVIVGGSSEYKINNKVVQLHEYSEELEKLGILIKARNFLVFQGAVESIAMKNPKERTALFEEISRSGDVAQEYDKRKKEMVKAEEDTQFNYHRKKNIAAERKEAKQEKEEADRYQRLKDEVVRAQVQLQLFKLYHNEVEIEKLNKELASKNKEIEKDKKRMDKVEDELKEKKKELGKMMREQQQIEKEIKEKDSELNQKRPQYIKAKENTSHKIKKLEAAKKSLQNAQKHYKKRKGDMDELEKEMLSVEKARQEFEERMEEESQSQGRDLTLEENQVKKYHRLKEEASKRAATLAQELEKFNRDQKADQDRLDLEERKKVETEAKIKQKLREIEENQKRIEKLEEYITTSKQSLEEQKKLEGELTEEVEMAKRRIDEINKELNQVMEQLGDARIDRQESSRQQRKAEIMESIKRLYPGSVYGRLIDLCQPTQKKYQIAVTKVLGKNMDAIIVDSEKTGRDCIQYIKEQRGEPETFLPLDYLEVKPTDEKLRELKGAKLVIDVIRYEPPHIKKALQYACGNALVCDNVEDARRIAFGGHQRHKTVALDGTLFQKSGVISGGASDLKAKARRWDEKAVDKLKEKKERLTEELKEQMKAKRKEAELRQVQSQAHGLQMRLKYSQSDLEQTKTRHLALNLQEKSKLESELANFGPRINDIKRIIQSREREMKDLKEKMNQVEDEVFEEFCREIGVRNIREFEEEKVKRQNEIA

SEQ ID NO: 28 - protein, 429 aa, Homo sapiens
KKRLEFENQKTRLGIQLDFEKNQLKEDQDKVHMWEQTVKKDENEIEKLKKEEQRHMKIIDETMAQLQDLKNQHLAKKSEVNDKNHEMEEIRKKLGGANKEMTHLQKEVTAIETKLEQKRSDRHNLLQACKMQDIKLPLSKGTMDDISQEEGSSQGEDSVSGSQRISSIYAREALIEIDYGDLCEDLKDAQAEEEIKQEMNTLQQKLNEQQSVLQRIAAPNMKAMEKLESVRDKFQETSDEFEAARKRAKKAKQAFEQIKKERFDRFNACFESVATNIDEIYKALSRNSSAQAFLGPENPEEPYLDGINYNCVAPGKRFRPMDNLSGGEKTVAALALLFAIHSYKPAPFFVLDEIDAALDNTNIGKVANYIKEQSTCNFQAIVISLKEEFYTKAESLIGVYPEQGDCVISKVLTFDLTKYPDANPNPNEQ

SEQ ID NO: 29 - DNA, 16 bp, Artificial
  Synthetic
gtatggttaatggctg

SEQ ID NO: 30 - DNA, 16 bp, Artificial
  Synthetic
atgccagccaaattgc

(19) United States
(12) Patent Application Publication
(10) Pub. No.: US 2020/0330606 A1
(43) Pub. Date: Oct. 22, 2020

(54) MODULATION OF STRUCTURAL MAINTENANCE OF CHROMOSOME-1 EXPRESSION

(71) Applicant: VALKYRIE THERAPEUTICS INC., LOS ANGELES, CA (US)

(72) Inventor: DAVID BERZ, LOS ANGELES, CA (US)

(21) Appl. No.: 16/090,043

(22) PCT Filed: Mar. 29, 2017
(86) PCT No.: PCT/US17/24785

§ 371 Date: Sep. 28, 2018

Related U.S. Application Data
(60) Provisional application No. 62/314,850, filed on Mar. 29, 2016.

(51) Int. Cl. A61K 47/68; C07K 16/40; C07K 16/18; G01N 33/483; A61K 47/69; A61P 35/00

(52) U.S. Cl. CPC A61K 47/6871; C07K 16/40; A61P 35/00; G01N 33/4833; A61K 47/6913; C07K 16/18

(57) ABSTRACT

Disclosed herein are antibody-conjugates and locked nucleic acid-modified oligonucleotides for modulating expression of one or more genes involved in one or more diseases. Compositions and kits comprising antibody-conjugates and/or locked nucleic acid-modified oligonucleotides for modulating expression of one or more genes involved in one or more diseases are disclosed. Methods of preventing and/or treating one or more diseases in a subject by modulating expression of one more genes by contacting cells and/or administering the compositions and kits to the subject are also disclosed.

### PRIORITY AND CROSS REFERENCE TO RELATED APPLICATIONS

This application claims the benefit of U.S. Provisional Application No. 62/314,850, filed on Mar. 29, 2016, which is hereby incorporated by reference in its entirety.

### SEQUENCE LISTING IN ELECTRONIC FORM

The present application is being filed along with an electronic Sequence Listing as an ASCII text file via EFS-Web. The electronic Sequence Listing is provided as a file entitled VALKY001WOSEQLIST.txt, created and last saved on Mar. 29, 2017, which is 27,264 bytes in size. The information in the electronic Sequence Listing is incorporated herein by reference in its entirety.

### BACKGROUND

### Field

The present disclosure is related to modulation of Structural Maintenance of Chromosome-1 (SMC1) expression. Some embodiments of the present disclosure are related to modulation of SMC1 expression in cancer.

### Description of the Related Art

Molecular strategies are being developed to modulate unwanted gene expression that either directly causes, participates in or aggravates a disease state, such as cancer. Such strategies involve inhibiting gene expression with oligonucleotides complementary in sequence to the messenger RNA of a deleterious target gene and/or using antibodies that modulate the function of one or more proteins associated with a disease state, such as cancer.

### SUMMARY

In some embodiments, an antibody-conjugate comprising an anti-SMC1 antibody is provided. In some embodiments of the antibody-conjugate, the antibody is conjugated to a cytotoxic molecule via a linker, wherein the antibody binds to one or more epitopes in an extracellular C-terminal region of SMC1. In some embodiments of the antibody-conjugate, the antibody binds to an epitope in the extracellular C-terminal region of SMC1 comprising residues 805-1233 of SMC1. In some embodiments of the antibody-conjugate, the epitope is selected from the group consisting of the sequences listed in Table 1. In some embodiments of the antibody-conjugate, the epitope is selected from the group consisting of SEQ ID NO: 11, SEQ ID NO: 12 and SEQ ID NO: 13. In some embodiments of the antibody-conjugate, the antibody is monoclonal or polyclonal. In some embodiments of the antibody-conjugate, the cytotoxic molecule is selected from the group consisting of calicheamicin, maytansinoids, auristatins, taxol, cytochalasin B, gramicidin D, ethidium bromide, emetine, mitomycin, vinca alkaloids, etoposide, tenoposide, vincristine, vinblastine, colchicin, doxorubicin, daunorubicin, dihydroxy anthracinedione, mitoxantrone, mithramycin, actinomycin D, methotrexate, adriamicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including and analogs, homologs, fragments and/or variants thereof, 1-dehydrotestosterone, glucocorticoids, procaine, tetracaine, lidocaine, propranolol, and puromycin, ricin, CC-1065, duocarmycins, diptheria toxin, venom (e.g., from snakes, amphibians, reptiles, fish, invertebrates, etc.), and analogs, homologs, fragments, and/or variants thereof, bismuth-213, astatine-211, radium-223, yttrium-90, iodine-131, samarium-153, strontium-89, lutetium-177, holmium-166, rhenium-186, rhenium-188, copper-67, promethium-149, gold-199, rhodium-105, bromine-77, indium-111, iodine-123, and iodine-125. In some embodiments, the antibody-conjugate specifically binds a cell that has surface expression of SMC1.

In some embodiments, an LNA-modified oligonucleotide comprising one or more LNAs is provided. In some embodiments, the LNA-modified oligonucleotide is complementary to an mRNA encoding SMC1, nd wherein the LNA-modified oligonucleotide binds the mRNA encoding SMC1 and targets the mRNA encoding SMC1 for degradation by an RNA silencing mechanism. In some embodiments, the length of the LNA-modified oligonucleotide is about 5 to about 50 nucleotides. In some embodiments, the sequence of the LNA-modified oligonucleotide is selected from the group consisting of 5′ GTATGGTTAATGGCTG 3′ (SEQ ID NO: 29) and 5′ ATGCCAGCCAAATTGC 3′ (SEQ ID NO: 30). In some embodiments, the number of LNAs in the LNA-modified oligonucleotide is about 1 to about 25. In some embodiments, one or more of the nucleotides in SEQ ID NO: 29 and SEQ ID NO: 30 are LNAs.

In some embodiments, a composition for preventing and/or treating a disease in a subject is provided. In some embodiments, the composition comprises an antibody-conjugate comprising an anti-SMC1 antibody, wherein the antibody is conjugated to a cytotoxic molecule via a linker, wherein the antibody binds to one or more epitopes in an extracellular C terminal region of SMC1, and an LNA-modified oligonucleotide comprising one or more LNAs, wherein the LNA-modified oligonucleotide is complementary to an mRNA encoding SMC1, and wherein the LNA-modified oligonucleotide binds the mRNA encoding SMC1 and targets the mRNA encoding SMC1 for degradation by an RNA silencing mechanism. In some embodiments of the composition, the antibody binds to an epitope in the extracellular C terminal region of SMC1 comprising residues 805-1233 of SMC1. In some embodiments of the composition, the epitope is selected from the group consisting of the sequences listed in Table 1. In some embodiments of the composition, the epitope is selected from the group consisting of SEQ ID NO: 11, SEQ ID NO: 12 and SEQ ID NO: 13. In some embodiments of the composition, the antibody is monoclonal or polyclonal. In some embodiments of the composition, the cytotoxic molecule is selected from the group consisting of calicheamicin, maytansinoids, auristatins, taxol, cytochalasin B, gramicidin D, ethidium bromide, emetine, mitomycin, vinca alkaloids, etoposide, tenoposide, vincristine, vinblastine, colchicin, doxorubicin, daunorubicin, dihydroxy anthracinedione, mitoxantrone, mithramycin, actinomycin D, methotrexate, adriamicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including and analogs, homologs, fragments and/or variants thereof, 1-dehydrotestosterone, glucocorticoids, procaine, tetracaine, lidocaine, propranolol, and puromycin, ricin, CC-1065, duocarmycins, diptheria toxin, venom (e.g., from snakes, amphibians, reptiles, fish, invertebrates, etc.), and analogs, homologs, fragments, and/or variants thereof, bismuth-213, astatine-211, radium-223, yttrium-90, iodine-131, samarium-153, strontium-89, lutetium-177, holmium-166, rhenium-186, rhenium-188, copper-67, promethium-149, gold-199, rhodium-105, bromine-77, indium-111, iodine-123, and iodine-125. In some embodiments of the composition, the antibody-conjugate specifically binds a cell that has surface expression of SMC1. In some embodiments of the composition, the length of the LNA-modified oligonucleotide is about 5 to about 50 nucleotides. In some embodiments of the composition, the sequence of the LNA-modified oligonucleotide is selected from the group consisting of 5′ GTATGGTTAATGGCTG 3′ (SEQ ID NO: 29) and 5′ ATGCCAGCCAAATTGC 3′ (SEQ ID NO: 30). In some embodiments of the composition, the number of LNAs in the LNA-modified oligonucleotide is about 1 to about 25. In some embodiments of the composition, one or more of the nucleotides in SEQ ID NO: 29 and SEQ ID NO: 30 are LNAs. In some embodiments of the composition, the preventing and/or treating is achieved by modulating an expression of SMC1 mRNA and SMC1 protein. In some embodiments of the composition, the disease is selected from the group consisting of breast cancer (e.g., triple negative breast cancer), breast adenocarcinoma, pancreatic adenocarcinoma, lung carcinoma, prostate cancer, hormone refractory prostate cancer, solid tumor malignancies such as colon carcinoma, non-small cell lung cancer (e.g., non-small cell lung cancer), anaplastic astrocytoma, glioma, glioblastoma (e.g., glioblastoma multiforme), bladder carcinoma, sarcoma, ovarian cancer, rectal hemangiopericytoma, pancreatic carcinoma, acute myeloid leukemia, cancer of large bowel, mesothelioma, stomach, pancreas, ovaries, melanoma, pancreatic cancer, colon cancer, and bladder cancer.

In some embodiments, a kit for preventing and/or treating a disease in a subject is provided. In some embodiments, the kit comprises any of the compositions described herein. In some embodiments of the kit, the disease is selected from the group consisting of breast cancer (e.g., triple negative breast cancer), breast adenocarcinoma, pancreatic adenocarcinoma, lung carcinoma, prostate cancer, hormone refractory prostate cancer, solid tumor malignancies such as colon carcinoma, non-small cell lung cancer (e.g., non-small cell lung cancer), anaplastic astrocytoma, glioma, glioblastoma (e.g., glioblastoma multiforme), bladder carcinoma, sarcoma, ovarian cancer, rectal hemangiopericytoma, pancreatic carcinoma, acute myeloid leukemia, cancer of large bowel, mesothelioma, stomach, pancreas, ovaries, melanoma, pancreatic cancer, colon cancer, and bladder cancer.

In some embodiments, a method of preventing and/or treating a disease in a subject is provided. In some embodiments, the method comprises performing a first assessment of the disease in the subject, providing any of the compositions described herein, administering any of the compositions described herein to the subject for a duration of time, performing a second assessment of the disease in the subject after the duration of time, wherein the second assessment of the disease indicates prevention and/or treatment of the disease in the subject after administering any of the compositions described herein to the subject for a duration of time, thereby preventing and/or treating the disease in the subject. In some embodiments of the method, the cell is a tumor cell, cancer cell, tumor stem cell, cancer stem cell, or a combination thereof. In some embodiments of the method, the antibody-conjugate is taken up by the cell. In some embodiments of the method, the cytotoxic molecule either arrests the growth of the cell or kills the cell. In some embodiments of the method, the LNA-modified oligonucleotide is formulated with a fusogenic or lipogenic component that allows an uptake of the LNA-modified oligonucleotide by a cell. In some embodiments of the method, the cell overexpresses an SMC1 mRNA. In some embodiments of the method, the LNA-modified oligonucleotide is complementary to the mRNA of SMC1, and wherein the LNA-modified oligonucleotide binds the SMC1 mRNA and modulates expression by an RNA silencing mechanism. In some embodiments of the method, the subject is a mammal, wherein the mammal is a human or a non-human. In some embodiments, the method further comprises providing one or more additional therapeutic agents. In some embodiments of the method, the one or more additional therapeutic agents is a PARP inhibitor. In some embodiments of the method, the one or more PARP inhibitor is selected from the group consisting of Niraparib (MK-4827), Iniparib (BSI 201), Talazoparib (BMN-673), Veliparib (ABT-888), Olaparib (AZD-2281), Rucaparib (AG014699, PF-01367338), CEP 9722, E7016, BGB-290, and 3-aminobenzamide. In some embodiments of the method, the one or more additional therapeutic agents is a platinum-based drug. In some embodiments of the method, the one or more platinum-based drug is selected from the group consisting of Cisplatin, Carboplatin, Oxaliplatin, Nedaplatin, Triplatin tetranitrate, Phenanthriplatin, Picoplatin, and Satraplatin. In some embodiments of the method, the one or more additional therapeutic agents potentiates the effect of any of the compositions described herein. In some embodiments of the method, the concertation of the antibody-conjugate in any of the compositions described herein ranges from about 1 nM to about 250 mM. In some embodiments of the method, the concertation of the LNA-modified oligonucleotide in any of the compositions described herein ranges from about 1 nM to about 250 mM.

In some embodiments, a method of diagnosing a state of a sample is provided. In some embodiments, the method comprises providing a sample wherein the sample is a cell suspension, tissue, biopsy or a combination thereof, providing an anti-SMC antibody, performing an immunohistochemical staining of the sample using the SMC1 antibody, determining a cellular localization of SMC1 in the sample, wherein a localization of SMC1 only in the nucleus is indicative of a normal state of the sample, and wherein a localization of SMC1 in the nucleus, the cytoplasmic and the plasma membrane is indicative of an abnormal state of the sample. In some embodiments of the method of diagnosing, the abnormal state of the sample is indicative of the presence of a disease. In some embodiments of the method of diagnosing the disease is selected from the group consisting of breast cancer (e.g., triple negative breast cancer), breast adenocarcinoma, pancreatic adenocarcinoma, lung carcinoma, prostate cancer, hormone refractory prostate cancer, solid tumor malignancies such as colon carcinoma, non-small cell lung cancer (e.g., non-small cell lung cancer), anaplastic astrocytoma, glioma, glioblastoma (e.g., glioblastoma multiforme), bladder carcinoma, sarcoma, ovarian cancer, rectal hemangiopericytoma, pancreatic carcinoma, acute myeloid leukemia, cancer of large bowel, mesothelioma, stomach, pancreas, ovaries, melanoma, pancreatic cancer, colon cancer, and bladder cancer.

### BRIEF DESCRIPTION OF THE DRAWINGS

FIG. 1 shows an embodiment of a schematic of the binding partners of Structural Maintenance of Chromosome-1 (SMC1)

FIG. 2 shows an embodiment of the amino acid sequence of human SMC1

FIG. 3 shows an embodiment of the amino acid sequence of the N-terminal human SMC1 representing residues 1-804.

FIG. 4 shows an embodiment of the amino acid sequence of the C-terminal human SMC1 representing residues 805-1233.

### DETAILED DESCRIPTION

Nucleotide-based molecular strategies are being developed to modulate unwanted gene expression that either directly causes, participates in or aggravates a disease state, such as cancer. One such strategy involves inhibiting gene expression with oligonucleotides complementary in sequence to the messenger RNA (mRNA) of a deleterious target gene. The mRNA is a copy of the coding (sense) DNA strand. Oligonucleotides that hybridize to the sense strand are called antisense oligonucleotides. Binding of antisense oligonucleotides to mRNA interferes with the translation process and consequently with gene expression. For example, antisense oligonucleotides have been used as anti-cancer agents by targeting, and down regulating, the activity of various oncogenes or proto-oncogenes. See e.g., U.S. Pat. No. 5,098,890 (MYB antisense for treating hematologic neoplasms, including use in bone marrow purging); International Patent Application WO 91/93260 (ABL antisense for treating myeloproliferative disorders); International Patent Application WO 92/19252 and Ratajczak et al., Proc Natl. Acad. Sci. USA 89, 1710-1714 (1992) (KIT for inhibiting malignant hematopoietic cell proliferation); International Patent Application W092/20348 and Melani et al., Cancer Res. 51; 2897-2901 (1991) (MYB antisense for inhibiting proliferation of colon cancer cells); International Patent Application WO92/22303 and Szcylick et al., Science 253, 562-565 (1991) (BCR-ABL antisense for inhibiting leukemia cell proliferation); and U.S. Pat. No. 5,087,617 (Bone marrow purging and in vivo therapy using antisense oligonucleotides to a variety of oncogenes or proto-oncogenes), which are incorporated by reference in their entireties.

In particular, various oligonucleotides have been investigated including single stranded and double stranded oligonucleotides, and their analogues. To be useful in in vivo applications, an oligonucleotide must have certain properties including the ability to penetrate a cell membrane, have good resistance to extra- and intracellular nucleases, have high affinity and specificity for the target and preferably have the ability to recruit endogenous enzymes such as RNAseH. An important property of oligonucleotides that underlies many of their potential therapeutic applications is their ability to recognize and hybridize specifically to complementary single stranded nucleic acids employing either Watson-Crick hydrogen bonding (A-T and G-C) or other hydrogen bonding schemes such as the Hoogsteen/reverse Hoogsteen mode. Affinity and specificity are properties commonly employed to characterize hybridization properties of the particular oligonucleotide. Affinity is a measure of the binding strength of the oligonucleotide to its complementary target (expressed as the thermostability (Tₘ) of the duplex). Each nucleotide base pair in the duplex adds to the thermostability and thus affinity increases with increasing size (number of nucleobases) of the oligonucleotide. Specificity is a measure of the ability of the oligonucleotide to discriminate between a fully complementary and a mismatched target sequence. In other words, specificity is a measure of the loss of affinity associated with mismatched nucleobase pairs in the target.

Locked nucleic acid (LNA) is a high-affinity and biologically stable RNA analog in which the normally flexible ribose sugar ring is fixed in a rigid conformation through a methylene 2′-O, 4′-C linkage. This fixed conformation brings substantial advantages to the design of effective RNA-binding molecules, and enables single-stranded LNA-modified oligonucleotides to have superior efficacies in vitro and in vivo in downregulating target mRNA expression as compared to oligonucleotides based on other chemistries, short interfering RNA (siRNA) and other antisense RNAs. The features that allow LNA-modified oligonucleotides to be a valuable therapeutic platform include, without limitations, unprecedented RNA binding affinity, detection of short RNA and DNA targets, superior specificity, single nucleotide discrimination, resistant to exonucleases and endonucleases resulting in high stability in vivo and in vitro, increased thermal stability of duplexes, safety, and ease of manufacture.

### Structural Maintenance of Chromosome-1 (SMC1)

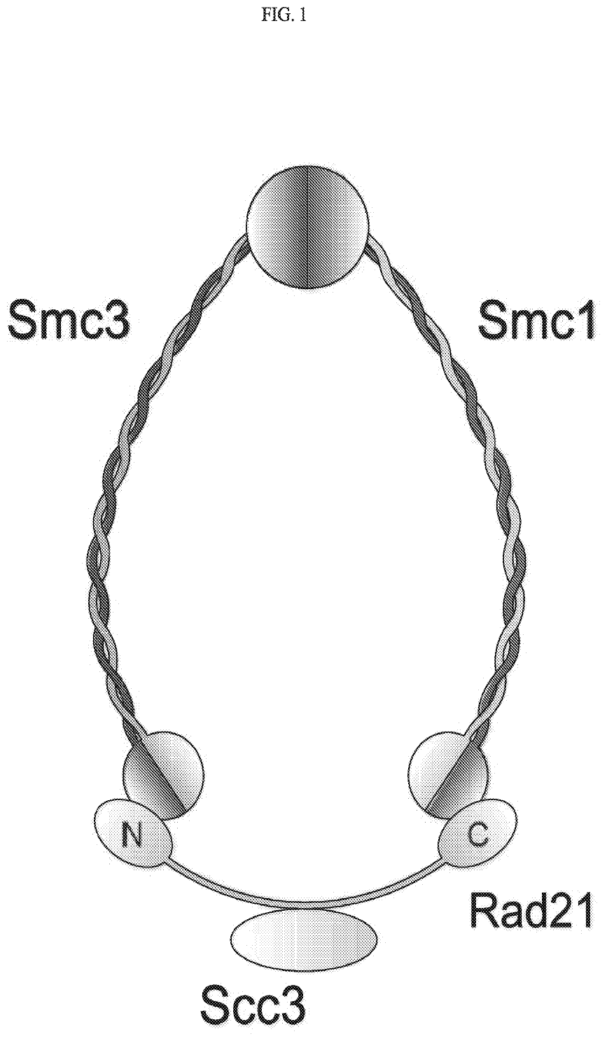

Human SMC1A (referred to herein as SMC1) is a well-described structural component of the cohesion complex and intimately involved with the partition of sister chromatids in mitosis. Overexpression of SMC1 is believed to be a predictor of poor prognosis in late stage colorectal cancer in humans. See, Wang et al, BMC Cancer. 2015 Mar. 4; 15: 90. doi: 10.1186/s12885-015-1085-4, which is hereby incorporated by reference in its entirety. Human SMC1 is a member of cohesion complex (FIG. 1), and along with SMC3, Rad21, SCC1/SCC2, it is known to be involved in chromosomal maintenance, DNA repair and gene expression. SMC1 binds with BRCA1 and together they are involved in the regulation of DNA damage response and cell cycle checkpoint-mediated DNA repair. In normal cells, SMC1 is described as a nuclear protein. The SMC1-SMC3 heterodimer constitutes an essential part of higher order complexes, which are involved in chromatin and DNA dynamics.

SMC1 is differentially overexpressed in tumor cells, cancer cells, tumor stem cells and cancer stem cells as compared to normal cells. For example, differential expression and mutations in SMC1 have been reported in many cancers including colon, acute myeloid leukemia, glioma and lung. Additionally, immunocytochemical, flow cytometry and cell fractionation studies have shown localization of SMC1 in the nucleus, cytoplasm and cell surface in tumor cells, cancer cells, tumor stem cells and cancer stem cells (e.g., triple negative breast cancer) as compared to predominantly nuclear localization in normal cells, suggesting that overexpression of SMC1 may play a role in carcinogenesis in cancers. For example, staining a lung cancer cell line (Am1010) with a monoclonal antibody against an epitope (SEQ ID NO: 11) of SMC1 showed surface localization of SMC in Am1010 cells (See, Example 2).

The present disclosure relates to therapeutic applications of anti-SMC1 antibodies and LNA-modified oligonucleotides against SMC1 in disease. The disclosure provides anti-SMC1 antibodies and LNA-modified oligonucleotides that modulate the expression of SMC1 thereby preventing and/or treating disease involving undesired cell growth (e.g., tumors and cancers). The disclosure also provides compositions and kits comprising antibodies and/or LNA-modified oligonucleotides for modulating SMC1 expression. Also provided are methods of preventing and/or treating one or more diseases comprising administration of compositions and kits comprising antibodies and/or LNA-modified oligonucleotides to a subject in need thereof to modulate SMC1 expression associated with undesired cell growth. In some embodiments, the LNA-modified oligonucleotides described herein target SMC1 mRNA for degradation via one or more RNA degradation and silencing mechanisms (e.g., RNAi). In some embodiments, the anti-SMC1 antibodies described herein target SMC1 protein for degradation via one or more protein degradation mechanisms (e.g., proteasome degradation).

Although the disclosure is related to human SMC1 (and its allelic variants, splice variants, protein variants, mutants, polymorphic variants, and other variants) and human cancer, one of ordinary skill in the art would appreciate that the antibody-conjugates and locked nucleic acid oligonucleotides for modulating expression of one or more genes involved in one or more diseases, compositions and kits comprising antibody-conjugates and/or locked nucleic acid oligonucleotides for modulating expression of one or more genes involved in one or more diseases, methods of preventing and/or treating one or more diseases in a subject by modulating expression of one more genes by contacting cells and/or administering the compositions and kits to the subject, and method of diagnosing a state of a sample can be extended to other human genes and other human disease, as well as for genes and diseases of other animals. Non-limiting examples of other genes include oncogenes, tumor suppressor genes, DNA repair genes, genes encoding tissue matrix proteins, genes encoding transporters, genes encoding signal transduction proteins, genes encoding cell growth and differentiation proteins, genes encoding proteins involved in biochemical reactions, genes encoding proteins involved in regulation of gene expression, etc. Non-limiting examples of other diseases include biochemical diseases, metabolic diseases, genetic diseases, parasitic diseases, congenital diseases, bacterial disease, viral diseases, autoimmune diseases, allergies, asthma, celiac disease, Crohn's disease, colitis, heart diseases, infectious diseases, liver diseases, neural diseases, etc.

### SMC1 Epitopes and Anti-SMC1 Antibody-Conjugates

SMC1 is differentially overexpressed in tumor cells, cancer cells, tumor stem cells and cancer stem cells as compared to normal cells. SMC1 localizes in the nucleus, cytoplasm and cell surface of tumor cells, cancer cells, tumor stem cells and cancer stem cells as compared to predominantly nuclear localization in normal cells.

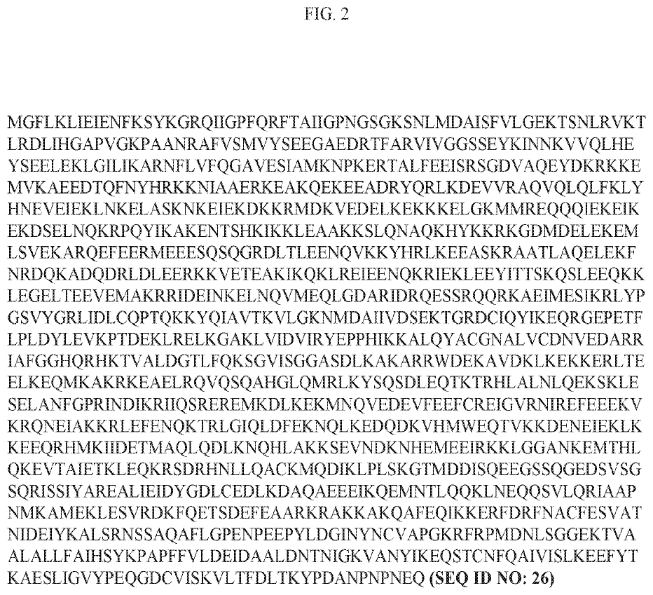

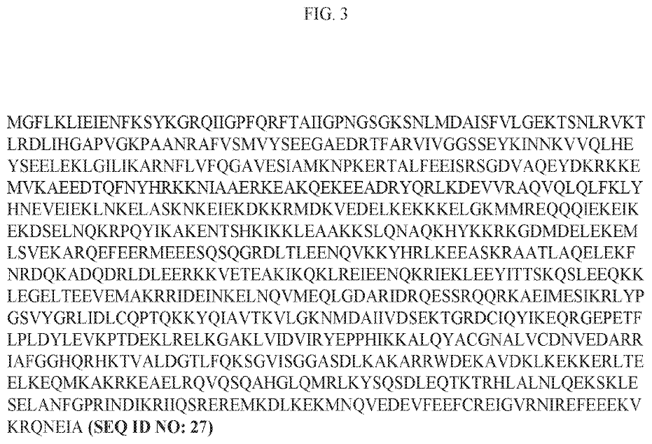

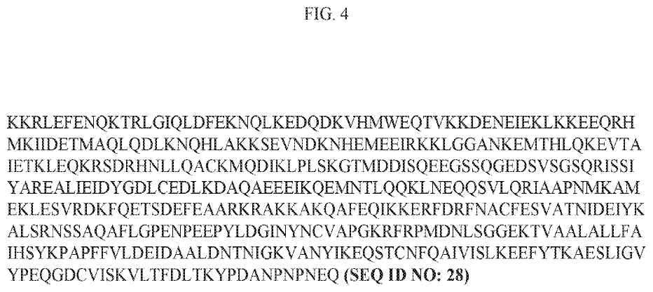

In some embodiments, the present disclosure is related to one or more SMC1 epitopes. The length of SMC1 (SEQ ID NO: 26; FIG. 2) is 1233 amino acids. Residues 1-804 (SEQ ID NO: 27; FIG. 3) comprise the intracellular and transmembrane domains. Binding partners to the N terminal domain(s) tether SMC1 intracellularly to the plasma membrane. Amino acid residues 805-1233 (SEQ ID NO: 28; FIG. 4)) of SMC1 are extracellular on the cell surface in tumor cells, cancer cells, tumor stem cells and cancer stem cells.

Residues 1-804 (SEQ ID NO: 27) of SMC1 represent the intracellular and transmembrane regions of SMC1. Thus, in some embodiments, one or more SMC1 epitopes are derived from the intracellular and/or transmembrane region of SMC1. SMC1 epitopes are derived from the intracellular and/or transmembrane region of SMC1. In some embodiments, one or more epitopes are derived from amino acid residues 1-804 (SEQ ID NO: 27) of SMC1. Residues 805-1233 (SEQ ID NO: 28) of SMC1 represent the extracellular (cell surface) region of SMC1 in tumor cells, cancer cells, tumor stem cells and cancer stem cells. Thus, in some embodiments, one or more SMC1 epitopes are derived from the region of SMC1 that is extracellular and/or exposed on the cell surface. In some embodiments, one or more epitopes are derived from amino acid residues 805-1233 (SEQ ID NO: 28) of SMC1.

In some embodiments, non-limiting examples of epitopes include the sequences provided in Table 1.

TABLE 1
Epitopes recognized by anti-SMC1 antibodies

| | SEQ | Corresponding |
|---|---|---|
| | ID | residues to |
| Sequence | NO | SEQ ID NO: 26 |
| EEDTQFNYHRKK | 1 | 179-190 |
| EKLNKELASKNK | 2 | 236-247 |
| TLEENQVKKYHR | 3 | 366-377 |
| LKEEASKRAA | 4 | 378-387 |
| KADQDRLDL | 5 | 401-409 |
| EERKKVETEAK | 6 | 410-420 |
| EIEENQKRIEK | 7 | 427-437 |
| DRQES SRQQRKA | 8 | 490-501 |
| TDEKLRELKGAK | 9 | 581-592 |
| RYEPPHIKKALQ | 10 | 599-610 |
| KKRLEFENQKTR | 11 | 805-816 |
| QDLKNQUILAKKS | 12 | 871-882 |
| TKYPDANPNPNE | 13 | 1221-1232 |
| MGFLKLIEIEN | 14 | 1-11 |
| FKSYKGR | 15 | 12-18 |
| ENFKSYKGRQII | 16 | 10-21 |
| QRFTAIIGPNGSGKSNL | 17 | 25-41 |
| NGSGKSNLMDAISFV | 18 | 34-48 |
| DAISFVLGEKTSNLRVKTLRD | 19 | 43-63 |
| LIHGAPVGKPAAN | 20 | 64-76 |
| PAANRAFVSMVYSEEGAED | 21 | 73-91 |
| DRTFARVIVGGSSEYKINNKVVQ | 22 | 91-113 |
| HEYSEELEKLGILIKARNFLVFQGAV | 23 | 115-140 |
| ESIAMICNPKERTA | 24 | 141-153 |
| LFEEISRSGDVAQEYD | 25 | 154-174 |

In preferred embodiments, one or more SMC1 epitopes are derived from the region of SMC1 that is extracellular and/or exposed on the cell surface, i.e., amino acid residues 805-1233 (SEQ ID NO: 28) of SMC1. In some embodiments, the size of the epitope ranges from about 5 amino acids to about 35 amino acids. In some embodiments, the size of the epitope ranges from about 5-10, 10-15, 15-20, 20-25, 25-30, 30-35 amino acids. In some embodiments, the size of the epitope is about 5, 10, 15, 20, 25, 30 or 35 amino acids, or within a range defined by any two of the aforementioned values. In some embodiments, the size of the epitope is 12 amino acids. In some embodiments, the epitope is of SEQ ID NO: 11. In some embodiments, the epitope is of SEQ ID NO: 12. In some embodiments, the epitope is of SEQ ID NO: 13. In a preferred embodiment, the epitope is of SEQ ID NO: 11.

In some embodiments, one or more variants of the one or more of epitopes provided in Table 1 are contemplated. In some embodiments, the epitope variants share at least 70% sequence identity with one or more of the epitopes listed in Table 1. In some embodiments, the epitope variants share about 70% or more sequence identity with one or more of the epitopes listed in Table 1. In some embodiments, the epitope variants share greater than at least 99% sequence identity with one or more of the epitopes listed in Table 1. In some embodiments, the epitope variants share about 70%, 75%, 80%, 85%, 90%, 95%, 96% 97%, 98%, 99% or greater that 99% sequence identity with one or more of the epitopes listed in Table 1, or within a range defined by any two of the aforementioned values.

As mentioned herein, SMC1 is differentially overexpressed in tumor cells, cancer cells, tumor stem cells and cancer stem cells as compared to normal cells. Additionally, immunocytochemical, flow cytometry and cell fractionation studies have shown localization of SMC1 in the nucleus, cytoplasm and cell surface in tumor cells, cancer cells, tumor stem cells and cancer stem cells as compared to predominantly nuclear localization in normal cells. Thus, in some embodiments, the antibody-conjugates provided herein specifically bind cells that have cell surface expression of SMC1. In some embodiments, the antibody-conjugates provided herein specifically bind tumor cells and tumor stem cells that have cell surface expression of SMC1. In some embodiments, the antibody-conjugates provided herein specifically bind cancer cell and cancer stem cells that have cell surface expression of SMC1.

In some embodiments, the present disclosure is related to one or more antibodies against one or more SMC1 epitopes (e.g., Table 1) and their variants disclosed herein. In some embodiments, the one or more antibodies is monoclonal. In some embodiments, the one or more antibodies is polyclonal. In some embodiments, the one or more antibodies binds to epitopes in residues 1-804 (SEQ ID NO: 27) of SMC1. In some embodiments, the one or more antibodies binds to epitopes in residues 805-1233 (SEQ ID NO: 28) of SMC1. In some embodiments, the one or more antibodies binds to epitopes in residues 1-804 (SEQ ID NO: 27) of SMC1 and residues 805-1233 (SEQ ID NO: 28) of SMC1. In some embodiments, the one or more antibodies that binds to epitopes in residues 1-804 (SEQ ID NO: 27) of SMC1 is monoclonal. In some embodiments, the one or more antibodies that binds to epitopes in residues 1-804 (SEQ ID NO: 27) of SMC1 is polyclonal. In some embodiments, the one or more antibodies that binds to epitopes in residues 805-1233 (SEQ ID NO: 28) of SMC1 is monoclonal. In some embodiments, the one or more antibodies that binds to epitopes in residues 805-1233 (SEQ ID NO: 28) of SMC1 is polyclonal.

In some embodiments, the one or more antibodies bind to epitope of SEQ ID NO: 11. In some embodiments, the one or more antibodies bind to epitope of SEQ ID NO: 12. In some embodiments, the one or more antibodies bind to epitope of SEQ ID NO: 13. In some embodiments, the one or more antibodies bind to epitope of SEQ ID NO: 11 is polyclonal. In a preferred embodiment, the one or more antibodies bind to epitope of SEQ ID NO: 11 is monoclonal. In some embodiments, the one or more antibodies bind to epitope of SEQ ID NO: 13 is monoclonal. In a preferred embodiment, the one or more antibodies bind to epitope of SEQ ID NO: 13 is polyclonal.

In some embodiments, the antibodies are from humans. In some embodiments, the antibodies are from non-human species. In some embodiments, the protein sequences of the antibodies from non-human species have been modified to increase their similarity to antibody variants produced naturally in humans. In some embodiments, the antibodies are humanized antibodies. In some embodiments, the one or more antibodies is IgA, IgD, IgE, IgG, IgM or a combination thereof. In some embodiments, the antibody is a single chain antibody, an antibody fragment that specifically binds to the target epitope, a monoclonal antibody, a single chain monoclonal antibody, a monoclonal antibody fragment that specifically binds to a target epitope, a chimeric antibody, a chimeric antibody fragment that specifically binds to the target epitope, a bispecific antibody, a domain antibody, or a domain antibody fragment that specifically binds to the target epitope. In alternative embodiments, the binding moiety (that specifically binds to the epitope) is not an antibody, but a peptide, polypeptide, aptamer, etc. that exhibits sufficient binding affinity and specificity to deliver a cytotoxic payload to a cell expressing the target epitope on the cell surface.

In some embodiments, one or more variants of one or more of the antibodies disclosed herein are contemplated. In some embodiments, the antibody variants share at least 70% sequence identity with the one or more antibodies that bind the epitopes listed in Table 1. The identity is either relative to the entire antibody sequence or relative to a subpart of the antibody sequence (e.g., one or more CDRs). In some embodiments, the antibody variants share about 70% or more sequence identity. In some embodiments, the antibody variants share greater than at least 99% sequence identity. In some embodiments, the antibody variants share about 70%, 75%, 80%, 85%, 90%, 95%, 96% 97%, 98%, 99% or greater that 99% sequence identity, or within a range defined by any two of the aforementioned values.

As used herein “K_{D}” refers to the binding affinity of the one or more antibodies and/or their variants to the one or more epitopes and/or their variants ranges. In some embodiment, the value of K_{D} ranges from about 10⁻⁷ M to about 10⁻¹⁰ M. In some embodiment, the value of K_{D} ranges from about 10⁻¹⁰ M to about 10⁻¹³ M. In some embodiment, the value of K_{D} is about 10⁻⁶, 10⁻⁷, 10⁻⁸, 10⁻⁹, 10⁻¹⁰, 10⁻¹¹, 10⁻¹², 10⁻¹³ or 10⁻¹⁴ M, or within a range defined any of the aforementioned values.

In some embodiments, the one or more antibodies provided herein is conjugated to a cytotoxic molecule via a linker (referred to herein as an antibody-conjugate).

In some embodiments, the cytotoxic molecule is a cytotoxic agent, chemotherapeutic agent, cytotoxic drug, toxins, radionuclides, etc. The cytotoxic molecule is detrimental to cells and may either arrest the growth of cells, kill the cells, alter their biochemistry and/or physiology such that they become susceptible to killing by one or more therapeutic agents, or a combination thereof. In some embodiments, the cytotoxic molecule prevents and/or inhibits one or more functions of cells. In some embodiments, the cytotoxic molecule can cause cell death by one or more of unraveling of structural fibers in the cell, causing irreparable DNA damage, apoptosis, autophagy, necrosis, necroptosis, pyroptosis, or caspase-independent cell death.

Non-limiting examples of cytotoxic molecules include calicheamicin, maytansinoids, auristatins, taxol, cytochalasin B, gramicidin D, ethidium bromide, emetine, mitomycin, vinca alkaloids, etoposide, tenoposide, vincristine, vinblastine, colchicin, doxorubicin, daunorubicin, dihydroxy anthracinedione, mitoxantrone, mithramycin, actinomycin D, methotrexate, adriamicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including and analogs, homologs, fragments and/or variants thereof, 1-dehydrotestosterone, glucocorticoids, procaine, tetracaine, lidocaine, propranolol, and puromycin, ricin, CC-1065, duocarmycins, diptheria toxin, venom (e.g., from snakes, amphibians, reptiles, fish, invertebrates, etc.), and analogs, homologs, fragments, and/or variants thereof.

In some embodiments, an antibody-conjugate comprises one or more radionuclides, for example, as described in ncbi.nlm.nih.gov/books/NBK11464/, which is incorporated by reference in its entirety. In some embodiments, an antibody-conjugate comprises an alpha-emitting radionuclide. In some embodiments, an antibody-conjugate comprises a beta-emitting radionuclide. In some embodiments, an antibody-conjugate comprises an Auger electron-emitting radionuclide. In some embodiments, an antibody-conjugate comprises a combination of an alpha-emitting radionuclide and a beta-emitting radionuclide. In some embodiments, an antibody-conjugate comprises a combination of an alpha-emitting radionuclide and an Auger electron-emitting radionuclide. In some embodiments, an antibody-conjugate comprises a combination of a beta-emitting radionuclide and an Auger electron-emitting radionuclide. In some embodiments, an antibody-conjugate comprises a combination of an alpha-emitting radionuclide, a beta-emitting radionuclide, and an Auger electron-emitting radionuclide.

Non-limiting examples of alpha-emitting radionuclides include bismuth-213, astatine-211, and radium-223. Non-limiting examples of beta-emitting radionuclides include yttrium-90, iodine-131, samarium-153, and strontium-89. Non-limiting examples of beta-emitting radionuclides include lutetium-177, holmium-166, rhenium-186, rhenium-188, copper-67, promethium-149, gold-199, and rhodium-105. Non-limiting examples of Auger electron-emitting radionuclides include bromine-77, indium-111, iodine-123, and iodine-125.

In some embodiments, the number of cytotoxic molecule conjugated per antibody can range from about 1 to about 100. In some embodiments, the number of cytotoxic molecule conjugated per antibody can range from about 100 to about 1000. In some embodiments, the number of cytotoxic molecule conjugated per antibody is about 1, 2, 5, 10, 15, 20, 25, 30, 25, 40, 45, 50, 55, 60, 65, 70, 75, 80, 85, 90, 95, 100, 200, 250, 500, 750 or 1000, or within a range defined by any two of the aforementioned values. In other embodiments, the number of cytotoxic compound molecule bound per target-binding agent (e.g., antibody) molecule can be determined spectroscopically by measuring the ratio of the absorbance at 280 nm and 252 nm. An average of about 0.5-about 20 cytotoxic compounds/antibody molecule(s) can be linked by known methods (see e.g., WO 2014134457, which is incorporated herein by reference). In one embodiment, the average number of linked cytotoxic compound per target-binding agent in the conjugate is about 0.5 to about 10, about 0.5 to 2 (e.g., 0.4, 0.5, 0.6, 0.7, 0.8, 0.9, 1.0, 1.1, 1.2, 1.3, 1.4, 1.5, 1.6, 1.7, 1.8, 1.9, 2.0, or 2.1), about 2 to about 8 (e.g. , 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, or 8.1), about 2.5 to about 7, about 3 to about 5, about 2.5 to about 5.0 (e.g. , about 2.5, about 2.6, about 2.7, about 2.8, about 2.9, about 3.0, about 3.1, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0), about 2.5 to about 4.0, about 3.0 to about 4.0, about 3.2 to about 4.2, or about 4.5 to 5.5 (e.g. , about 4.5, about 4.6, about 4.7, about 4.8, about 4.9, about 5.0, about 5.1, about 5.2, about 5.3, about 5.4, or about 5.5), or within a range defined by any two of the aforementioned values.

As SMC1 is specifically expressed on the surface by tumor cells, tumor stem cells, cancer cells, and/or cancer stem cells, the antibody-conjugates disclosed herein are specifically bound and taken up by tumor cells, tumor stem cells, cancer cells, and/or cancer stem cells. Thus, the antibody-conjugates disclosed herein specifically and selectively kill tumor cells, tumor stem cells, cancer cells, and/or cancer stem cells of various histological types while sparing the normal cells. Non-limiting examples of histological types of tumor/cancer include carcinoma, sarcoma, myeloma, leukemia, lymphoma, and mixed types.

In some embodiments, the antibody-conjugates disclosed herein specifically and selectively kill tumor cells of various histological types. In some embodiments, the antibody-conjugates disclosed herein specifically and selectively kill tumor stem cells of various histological types. In some embodiments, the antibody-conjugates disclosed herein specifically and selectively kill cancer cells of various histological types. In some embodiments, the antibody-conjugates disclosed herein specifically and selectively kill cancer stem cells of various histological types.

In some embodiments, the antibody-conjugate comprises one or more linkers that link the antibody to the cytotoxic molecule. In some embodiments, the linker is attached to a cysteine residue on the antibody. In some embodiments, the linker is attached to a lysine residue on the antibody. In some embodiments, the linker is attached to a combination of cysteine and lysine residues on the antibody.

In some embodiments, the linker is cleavable allowing separation of the antibody and the cytotoxic molecule. In some embodiments, the linker is cleaved intracellularly. In some embodiments, the linker is cleaved extracellularly. In some embodiments, the linker is cleaved both intracellularly and extracellularly. In some embodiments, the linker is partially cleaved extracellularly and partially cleaved intracellularly. In some embodiments, the linker is partially cleaved extracellularly and completely cleaved intracellularly. In some embodiments, initial cleavage of the linker occurs extracellularly and final cleavage occurs intracellularly. In some embodiments, the linker is non-cleavable.

Non-limiting examples of cleavable linkers include hydrazone linkers, disulfide-based linkers and peptide linkers. In some embodiments, disulfide-based linkers are selectively broken down inside the tumor cell. In some embodiments, disulfide-based linkers are selectively broken down intracellularly because of higher intracellular concentration of thiols. In some embodiments, peptide linkers are selectively broken down intracellularly by intracellular enzymes. Non-limiting examples of non-cleavable linkers include thioether linkers, and PEG4Mal linker.

As one of ordinary skill in the art would appreciate, within the scope of this disclosure for generating antibody-conjugates are other linker types, conjugation chemistries, conjugation sites on antibody, etc., Non-limiting examples are disclosed in WO 2014134457, WO 2009097397, U.S. Pat. Nos. 9,156,854 and 9,388,408, scripps.edu/baran/images/grpmtgpdf/Sella_May_14.pdf, ncbi.nlm.nih.gov/pmc/articles/PMC4365093/, nature.com/nchem/journal/v8/n2/full/nchem.2415.html, princeton.edu/chemistry/macmillan/group-meetings/CL_Antibody-Drug-Conjugates.pdf, which are hereby incorporated by reference in their entireties.

In some embodiments, the anti-SMC1 antibody-conjugates described herein preferentially bind SMC1 expressed on the cell surface of cancer cells and cancer stem cells. The anti-SMC1 antibody-conjugates bound SMC1 on the surface of cancer cells and cancer stem cells are taken up and degraded by one or more mechanisms of protein uptake and degradation known in the art. Non-limiting examples include receptor-mediated endocytosis, clathrin-mediated endocytosis, endocytosis at caveolae, potocytosis, macropinocytosis, ATP-powered transport, and phagocytosis. Once inside the cell, the SMC1 taken up from the cell surface is degraded within the cell by one or more cellular proteases and by one or more protein degradation mechanisms known in the art. Non-limiting examples of proteases include cysteine protease, serine protease, threonine protease, aspartic protease, glutamic protease, metalloprotease and asparagine peptide lyase. Non-limiting examples of protein degradation mechanisms include proteasome, ubiquitin, sumoylation, and autophagy. The degradation of SMC1 results in downregulation of expression of SMC1 in the cancer cells and cancer stem cells.

An efficacy of the antibody-conjugate can be measured by quantifying the decrease in SMC1 expression by the anti-SMC1 antibody-conjugate. Quantification of protein expression is performed using Western blotting, flowcytometry, or other tools that are well-known in the art.

In some embodiments, an efficacy of the antibody-conjugate is ranges from about 75% to about 100%. The efficacy of the antibody-conjugate ranges is about 50, 55, 60, 65, 70, 75, 80, 85, 90, 91, 92, 93, 94, 95, 96, 97, 98, 99 or about 100%, or within a range defined by any two of the aforementioned values. In other words, the expression of SMC1 is decreased by the antibody-conjugate by about 75% to about 100%. In some embodiments, the expression of SMC1 is decreased by the antibody-conjugate by about 50, 55, 60, 65, 70, 75, 80, 85, 90, 91, 92, 93, 94, 95, 96, 97, 98, 99 or about 100%, or within a range defined by any two of the aforementioned values.

In some embodiments, a nonspecific effect (off-target effect or bystander effect) is less than about 10%. In some embodiments, a nonspecific effect (off-target effect or bystander effect) ranges from about 0% to about 10%. In some embodiments, a nonspecific effect (off-target effect or bystander effect) is about 0, 1, 2, 3, 4, 5, 6, 7, 8, 9 or 10%, or within a range defined by any two of the aforementioned values.

### LNA-Modified Oligonucleotides

As used herein, the term “LNA-modified oligonucleotide” includes any oligonucleotide either fully or partially modified with one or more LNA monomers. According to a preferred embodiment of the present invention, an LNA modified antisense oligonucleotide is designed to be specific for the mRNA of a gene involved in causing, and/or aggravating a disease. In some embodiments, this is be achieved by reducing or inhibiting the expression of the gene(s) involved in causing and/or aggravating disease and/or by inducing or increasing the expression of normally lowly expressed or unexpressed gene(s), the expression of which may mitigate and/or cure the disease. Such approaches are described, for example, in Mollegaard et al. Proc. Natl. Acad. Sci. USA, 1994, 91(9), 3892-3895, which is hereby incorporated by reference in its entirety, non-limiting examples of which include modulation, induction or increases in the expression of a target gene may be achieved by directing the antisense oligonucleotide against the mRNA of a gene that encodes a natural repressor of the target gene, by designing the antisense oligonucleotide in such a way that binding to its complementary sequence in the target mRNA will lead to an increase in target mRNA half-life and expression, or by using an oligonucleotide that can strand invade dsDNA to form a complex that can function as an initiation point for transcription of a downstream gene.

In some embodiments, the LNA-modified oligonucleotides described herein target SMC1 mRNA for degradation one or more RNA degradation and silencing mechanisms (e.g., RNAi based on microRNA (miRNA), small interfering RNA (siRNA), piwi-interacting RNA (piRNA), and repeat associated small interfering RNA (rasiRNA)).

SMC1 mRNA levels in tumor and/or cancer cells are about 1.5-fold to about 1500-fold fold higher as compared to normal cells. As tumor and/or cancer cells express more SMC1 mRNA as compared to normal cells, the does and/or amount of LNA-modified oligonucleotides can be titrated such that LNA-modified oligonucleotides would selectively kill cancer cells while sparing normal cells. Thus, in some embodiments, LNA-modified oligonucleotides are used at a concentration of about 1nM to about 250 mM. The LNA-modified oligonucleotides are toxic to tumor and/or cancer cells at a concentration of about 1 nM to about 250 mM.

In some embodiments, one or more oligonucleotides comprising locked nucleic acids (LNAs) are provided as for example disclosed in U.S. Pat. No. 9,388,408 B2, which is hereby incorporated by reference in its entirety. In some embodiments, the one or more LNA-modified oligonucleotides comprise an antisense sequence to an mRNA. In some embodiments, the one or more LNA-modified oligonucleotides consist essentially of an antisense sequence to an mRNA. In some embodiments, LNA-modified oligonucleotides consist of an antisense sequence to an mRNA. In some embodiments, the one or more LNA-modified oligonucleotides is complementary to an mRNA sequence such that it hybridize to the endogenous mature mRNA under physiological conditions.

In some embodiments, the LNA-modified oligonucleotides are complementary to the mRNA of SMC1. In some embodiments, the LNA-modified oligonucleotides are complementary to the mRNA of one or more other genes encoding proteins of the cohesion complex. For example, in some embodiments, the one or more other genes are SMC2 and SMC3 of the cohesion complex, multidrug transporter genes (e.g., MDR-1, MDR-2, or MGR-2), or a combination thereof. In some embodiments, an LNA-modified oligonucleotide is employed that enables effective modulation of a specific gene(s), for example, genes involved in human or mammalian diseases. For example, in some embodiments, the LNA-modified oligonucleotides is specific for cancer-causing genes such as for instance including breast, ovarian, mesothelioma, colon, acute myeloid leukemia, glioma, glioblastoma multiforme and lung cancer and modulate the expression of cancer-causing genes such as for instance including breast, ovarian, mesothelioma, colon, acute myeloid leukemia, glioma, glioblastoma multiforme and lung cancer.

In some embodiments, the disease is caused by either inherited or acquired genetic disorders or is one in which a normal gene product is involved in a pathophysiological process. In some embodiments, an LNA-modified oligonucleotide may be used against protein coding genes as well as non-protein coding genes. Examples of non-protein coding genes include genes that encode ribosomal RNAs, transfer RNAs, small nuclear RNAs, small cytoplasmic RNAs, telomerase RNA. Other types of RNA are also within the scope of this disclosure non-limiting examples of which include RNAs involved in protein synthesis, RNAs involved in post-transcriptional modification, RNAs involved in DNA replication, regulatory RNAs, and parasitic RNAs.

In some embodiments, the one or more LNA-modified oligonucleotides is specific and unique to an mRNA of one gene such that the one or more LNA-modified oligonucleotides does not hybridize to mRNAs from other genes. In some embodiments, the one or more LNA-modified oligonucleotides are completely (i.e., 100%) complementary an mRNA. In some embodiments, the one or more LNA-modified oligonucleotides is partially complementary to an mRNA. For example, in some embodiments, the one or more LNA-modified oligonucleotides is about 75% to about 99% complementary to an mRNA. In some embodiments, the one or more LNA-modified oligonucleotides is at least about 75%, 80%, 85%, 90%, 95%, 96%, 97%, 98% or 99% complementary to an mRNA, or within a range defined by any two of the aforementioned values. Owing to superior specificity and ability discriminate single nucleotide differences, an LNA-modified oligonucleotide need not be 100% complementary to its target. Thus, one or more LNA-modified oligonucleotide is about 75%, 80%, 85%, 90%, 95%, 96%, 97%, 98% or 99% complementary to an mRNA, or within a range defined by any two of the aforementioned values.

In some embodiments, the length of the LNA-modified oligonucleotide ranges from about 5 to about 50 nucleotides. In some embodiments, the LNA-modified oligonucleotide is about 5 to 10 nucleotides in length. In some embodiments, the LNA-modified oligonucleotide is about 10 to 20 nucleotides in length. In some embodiments, the LNA-modified oligonucleotide is about 20 to 30 nucleotides in length. In some embodiments, the LNA-modified oligonucleotide is about 30 to 40 nucleotides in length. In some embodiments, the LNA-modified oligonucleotide is about 40 to 50 nucleotides in length. In some embodiments, the LNA-modified oligonucleotide is about 5, 10, 15, 20, 25, 30, 35, 40, 45 or 50 nucleotides in length, or within a range defined by any two of the aforementioned values.

In some embodiments, the percent of LNAs in the LNA-modified oligonucleotides ranges from about 0.02% to about 50%. In some embodiments, the percent of LNAs in the LNA-modified oligonucleotides is about 0.02, 0.05, 0.1, 0.2, 0.5, 1, 5, 10, 15, 20, 25, 30, 35, 40, 45 or 50%, or within a range defined by any two of the aforementioned values. In some embodiments, the LNA-modified oligonucleotide comprises an LNA at position n, n+1, n+2 . . . n+m or a combination thereof, where n is the first nucleotide position and m is the last nucleotide position. In some embodiments, the LNA-modified oligonucleotide comprises at most 10 LNA. In some embodiments, the LNA-modified oligonucleotide comprises 10 or fewer LNA. In some embodiments, the LNA-modified oligonucleotide comprises at most 10, 9, 8, 7, 6, 5, 4, 3, 2 or 1 LNA. In some embodiments, the LNA-modified oligonucleotide comprises at most 5 contiguous LNA. In some embodiments, the LNA-modified oligonucleotide comprises 5 or fewer contiguous LNAs. In some embodiments, the LNA-modified oligonucleotide comprises at most 5, 4, 3 or 2 contiguous LNA. In some embodiments, LNA is located at the 5′ end. In some embodiments, LNA is located at the 5′ end and internally. In some embodiments, LNA is located at the 3′ end. In some embodiments, LNA is located at the 3′ end and internally. In some embodiments, LNA is located at the 5′ and 3′ ends. In some embodiments, LNA is located at the 5′ and 3′ ends and internally.

In some embodiments, the SMC1 LNA-modified oligonucleotide is 5′ GTATGGTTAATGGCTG 3′ (SEQ ID NO: 29). In some embodiments, the SMC1 LNA-modified oligonucleotide is 5′ ATGCCAGCCAAATTGC 3′ (SEQ ID NO: 30). In some embodiments, one or more of the nucleotides in SEQ ID NO: 29 are LNAs. In some embodiments, one or more of the nucleotides in SEQ ID NO: 30 are LNAs. In some embodiments, one or more nucleotides at any position in SEQ ID NO: 29 are LNAs. In some embodiments, one or more nucleotides at any position in SEQ ID NO: 30 are LNAs. In some embodiments, one nucleotide in SEQ ID NO: 29 and one nucleotide in SEQ ID NO: 30 is an LNA. In some embodiments, two nucleotides in SEQ ID NO: 29 and two nucleotides in SEQ ID NO: 30 are LNAs. In some embodiments, three nucleotides in SEQ ID NO: 29 and three nucleotides in SEQ ID NO: 30 are LNAs. In some embodiments, four nucleotides in SEQ ID NO: 29 and four nucleotides in SEQ ID NO: 30 are LNAs. In some embodiments, five nucleotides in SEQ ID NO: 29 and five nucleotides in SEQ ID NO: 30 are LNAs. In some embodiments, six nucleotides in SEQ ID NO: 29 and six nucleotides in SEQ ID NO: 30 are LNAs. In some embodiments, seven nucleotides in SEQ ID NO: 29 and seven nucleotides in SEQ ID NO: 30 are LNAs. In some embodiments, eight nucleotides in SEQ ID NO: 29 and eight nucleotides in SEQ ID NO: 30 are LNAs. In some embodiments, nine nucleotides in SEQ ID NO: 29 and nine nucleotides in SEQ ID NO: 30 are LNAs. In some embodiments, 10 nucleotides in SEQ ID NO: 29 and 10 nucleotides in SEQ ID NO: 30 are LNAs. In some embodiments, 11 nucleotides in SEQ ID NO: 29 and 11 nucleotides in SEQ ID NO: 30 are LNAs. In some embodiments, 12 nucleotides in SEQ ID NO: 29 and 12 nucleotides in SEQ ID NO: 30 are LNAs. In some embodiments, 13 nucleotides in SEQ ID NO: 29 and 13 nucleotides in SEQ ID NO: 30 are LNAs. In some embodiments, 14 nucleotides in SEQ ID NO: 29 and 14 nucleotides in SEQ ID NO: 30 are LNAs. In some embodiments, 15 nucleotides in SEQ ID NO: 29 and 15 nucleotides in SEQ ID NO: 30 are LNAs. In some embodiments, 16 nucleotides in SEQ ID NO: 29 and 16 nucleotides in SEQ ID NO: 30 are LNAs.

In some embodiments, the LNA-modified oligonucleotides described herein comprise one or more non-locked nucleic acids. In some embodiments, at least one of the non-locked nucleotides is 2′ deoxy. In some embodiments, at least one of the non-locked nucleotides is 2′ 0-alkyl. In some embodiments, at least one of the non-locked nucleotides is 2′ halo. In some embodiments, at least one of the non-locked nucleotides is 2′ deoxy, 2′ 0-alkyl, 2′ halo or a combination thereof. In some embodiments, all of the non-locked nucleotides are either 2′ deoxy, 2′ 0-alkyl, or 2′ halo, or a combination thereof. In preferred embodiments, the LNA-modified oligonucleotides are designed such that they do not form hairpins, helices, pseudoknots, stem-loop structures or other secondary, tertiary or quaternary structures. In preferred embodiments, the LNA-modified oligonucleotides maintain their primary structure.

In some embodiments, target genes may be single-stranded or double-stranded DNA or RNA; however, single-stranded DNA or RNA targets are preferred. It is understood that the target to which the antisense oligonucleotides of the invention are directed include allelic forms of the targeted gene and the corresponding mRNAs including splice variants. There is guidance in the literature for selecting particular sequences for antisense oligonucleotides given a knowledge of the sequence of the target polynucleotide, e.g., Peyman and Ulmann, Chemical Reviews, 90:543-584, 1990; Crooke, Ann. Rev. Pharmacal. Toxicol., 32:329-376 (1992); and Zamecnik and Stephenson, Proc Natl. Acad. Sci., 75:280-284 (1974), which are incorporated by reference in their entireties. The sequences of antisense compounds may be selected such that the G-C content is at least 60%. Some mRNA targets include the 5′ cap site, fRNA primer binding site, the initiation codon site, the mRNA donor splice site, and the mRNA acceptor splice site, e.g., Goodchild et al., U.S. Pat. No. 4,806,463, which are incorporated by reference in its entirety.

Where the target polynucleotide comprises a mRNA transcript, oligonucleotides complementary to and hybridizable with any portion of the transcript are, in principle, effective for inhibiting translation, and capable of inducing the effects herein described. Without being bound by any theory, it is believed that translation is most effectively inhibited by blocking the mRNA at a site at or near the initiation codon. Thus, in some embodiments, LNA-modified oligonucleotide are complementary to the 5′ region of mRNA transcript. In other embodiments, LNA-modified oligonucleotides are complementary to the 5′ region of an mRNA transcript, including the initiation codon (the first codon at the 5′ end of the translated portion of the transcript), or codons adjacent to the initiation codon.

While LNA-modified oligonucleotides complementary to the coding region of mRNA transcripts may be preferred, particularly the region including the initiation codon, it will be appreciated by one of ordinary skill in the art that LNA-modified oligonucleotides complementary to untranslated portions of mRNA can also be designed. For example, in some embodiments, LNA-modified oligonucleotides are complementary to mRNA sequences contained in, or extending into, the 5′ untranslated region, 3′ untranslated region, or a combination thereof. Without being bound by any theory, LNA-modified oligonucleotides complementary to the 3′-untranslated region may be particularly useful modulating mRNA half-life thereby potentially regulating its expression/translation.

In some embodiments, LNA-modified oligonucleotides can be designed from any part of a precursor mRNA and/or mature mRNA including, but not limited to, the cap, 5′ untranslated region, coding sequence, intron, exon, 3′-untranslated region, and/or poly-A tail. In some embodiments, one or more LNA-modified oligonucleotides are designed against any part of a precursor mRNA and/or mature mRNA of human SMC1 mRNA. In some

### Compositions

In some embodiments, compositions for preventing and/or treating one or more diseases in a subject are provided. In some embodiments, compositions comprise one or more antibody-conjugates disclosed herein. In some embodiments, compositions comprise one or more LNA-modified oligonucleotides disclosed herein. In some embodiments, compositions comprise a combination of one or more antibody-conjugates and LNA-modified oligonucleotides disclosed herein. It will be understood by one of ordinary skill in the art that any embodiment of antibody-conjugate provided herein can be combined with any embodiment of LNA-modified nucleotide provided herein.

In some embodiments, methods of preventing and/or treating of treatment of one or more diseases in a subject are provided. In some embodiments, the methods comprise administering to subject in need thereof the one or more compositions disclosed herein. In some embodiments, the methods comprise administering the one or more antibody-conjugates disclosed herein. In some embodiments, the methods comprise administering the one or more LNA-modified oligonucleotides disclosed herein. In some embodiments, the methods comprise administering a combination of one or more antibody-conjugates and LNA-modified oligonucleotides disclosed herein.

In some embodiments, the one or more diseases is cancer. In some embodiments, the tumor and/or cancer may be selected from one or more of the following, including without limitation, breast cancer (e.g., triple negative breast cancer), breast adenocarcinoma, pancreatic adenocarcinoma, lung carcinoma, prostate cancer, hormone refractory prostate cancer, solid tumor malignancies such as colon carcinoma, non-small cell lung cancer (e.g., non-small cell lung cancer), anaplastic astrocytoma, glioma, glioblastoma (e.g., glioblastoma multiforme), bladder carcinoma, sarcoma, ovarian cancer, rectal hemangiopericytoma, pancreatic carcinoma, advanced cancer, acute myeloid leukemia, cancer of large bowel, mesothelioma, stomach, pancreas, ovaries, melanoma, pancreatic cancer, colon cancer, and bladder cancer.

In some embodiments, the concentration of antibody-conjugate in the compositions ranges from about 1 nM to about 1 mM. In some embodiments, the concentration of antibody-conjugate in the compositions ranges from about 1 mM to about 250 mM. In some embodiments, the concentration of antibody-conjugate in the compositions is about 1 nM, 100 nM, 500 nM, 1 μM, 100 μM, 500 μM, 1 mM, 100 mM or 250 mM, or within a range defined by any two of the aforementioned values.

In some embodiments, the concentration of LNA-modified oligonucleotides in the compositions ranges from about 1 nM to about 1 mM. In some embodiments, the concentration of LNA-modified oligonucleotides in the compositions ranges from about 1 mM to about 250 mM. In some embodiments, the concentration of LNA-modified oligonucleotides in the compositions is about 1 nM, 100 nM, 500 nM, 1 μM, 100 μM, 500 μM, 1 mM, 100 mM or 250 mM, or within a range defined by any two of the aforementioned values.

In some embodiments of the compositions comprising a combination of antibody-conjugate and LNA-modified oligonucleotide, the concentration of antibody ranges from about 1 nM to about 1 mM, and the concertation of LNA-modified oligonucleotide ranges from about 1 nM to about 1 mM. In some embodiments of the compositions comprising a combination of antibody-conjugate and LNA-modified oligonucleotide, the concentration of antibody ranges from about 1 mM to about 250 mM, and the concertation of LNA-modified oligonucleotide ranges from about 1 mM to about 250 mM. In some embodiments of the compositions comprising a combination of antibody-conjugate and LNA-modified oligonucleotide, the concentration of antibody-conjugate in the compositions is about 1 nM, 100 nM, 500 nM, 1 μM, 100 μM, 500 μM, 1 mM, 100 mM or 250 mM, or within a range defined by any two of the aforementioned values, and the concentration of LNA-modified oligonucleotides in the compositions is about 1 nM, 100 nM, 500 nM, 1 μM, 100 μM, 500 μM, 1 mM, 100 mM or 250 mM, or within a range defined by any two of the aforementioned values.

In some embodiments, the method comprises administering antibody and/or antibody-drug conjugate at a dose of about 1 mg/kg to about 100 mg/kg. In some embodiments, the method comprises administering antibody and/or antibody-drug conjugate at a dose of about 10 mg/kg to about 500 mg/kg. In some embodiments, the method comprises administering antibody and/or antibody-drug conjugate at a dose of about 1, 5, 10, 50, 100, 200, 350, 300, 350, 400, 450 or 500 mg/kg, or within a range defined by any two of the aforementioned values.

In some embodiments, the method comprises administering LNA-modified oligonucleotides at a dose of about 1 mg/kg to about 100 mg/kg. In some embodiments, the method comprises administering LNA-modified oligonucleotides at a dose of about 10 mg/kg to about 500 mg/kg. In some embodiments, the method comprises administering LNA-modified oligonucleotides at a dose of about 1, 5, 10, 50, 100, 200, 350, 300, 350, 400, 450 or 500 mg/kg, or within a range defined by any two of the aforementioned values.

In some embodiments, the method comprise administering a combination of antibody-drug conjugate at a dose of about 1 mg/kg to about 100 mg/kg and LNA-modified oligonucleotide at a dose of about 1 mg/kg to about 100 mg/kg. In some embodiments, the method comprise administering a combination of antibody-drug conjugate at a dose of about 10 mg/kg to about 500 mg/kg and LNA-modified oligonucleotide at a dose of about 10 mg/kg to about 500 mg/kg. In some embodiments, the method comprises administering a combination of antibody-drug conjugate at a dose of about 1, 5, 10, 50, 100, 200, 350, 300, 350, 400, 450 or 500 mg/kg, or within a range defined by any two of the aforementioned values, and LNA-modified oligonucleotide at a dose of about 1, 5, 10, 50, 100, 200, 350, 300, 350, 400, 450 or 500 mg/kg, or within a range defined by any two of the aforementioned values.

In some embodiments, the one or more LNA-modified oligonucleotides is comprised in macromolecular assemblies. Non-limiting examples include liposomes, nanoparticles, and micelles, formulated for delivery to a cell and/or a patient. In some embodiments, the macromolecular assemblies comprise one or more fusogenic and/or lipophilic molecules to initiate cellular membrane penetration. Non-limiting examples are described, for example, in U.S. Pat. No. 9,388,408 B2, which is hereby incorporated by reference in its entirety. Alternatively, the LNA-modified oligonucleotides may comprise a lipophilic group to achieve cellular delivery. Non-limiting examples are described in U.S. Pat. No. 9,388,408 B2, which is hereby incorporated by reference in its entirety. In some embodiments, the LNA-modified oligonucleotides further comprise a hydrophilic group to target the LNA-modified oligonucleotides to particular cell, organs and/or tissues. For example, in some embodiments, the LNA-modified oligonucleotides are conjugated to a sugar moiety, (e.g., mannose-6-phosphate) and/or an amino sugar (e.g., N-acetyl glucosamine).

In some embodiments, the antibody-conjugates and LNA-modified oligonucleotides described herein are formulated as compositions prepared in a form appropriate for an intended pharmaceutical and/or therapeutic application. In some embodiments, such compositions are free of pyrogens as well as other impurities that could be harmful to humans and/or animals. Non-limiting examples of compositions formulated for pharmaceutical and/or therapeutic application include colloidal dispersion systems, macromolecule complexes, nanocapsules, nanoparticles, microspheres, beads, and lipid-based systems including oil-in-water emulsions, fat emulsions, micelles, mixed micelles, and liposome (i.e., an artificial membrane vesicle). Non-limiting examples of commercially available fat emulsions suitable for intravenously delivering LNA-modified oligonucleotides include Intralipid®, Liposyn®, Liposyn® II, Liposyn® III, Nutrilipid, and the like. Other non-limiting examples are disclosed in U.S. Pat. No. 9,388,408 B2, which is hereby incorporated by reference in its entirety.

In some embodiments, the delivery of the composition is in vitro. In some embodiments, the delivery of the composition is in vivo. In some embodiments, the components of the composition are co-administered. In some embodiments, the components of the composition are administered separately and sequentially.

Oftentimes, a plurality of abnormalities involving genes associated with cancer add up before a tumor and/or a cancer develops. In these cases, combinations of LNA-modified antisense oligonucleotides specific for the different genes may be administered simultaneously or separately. In some embodiments, more than one LNA-modified oligonucleotide is. In some embodiments, several LNA-modified oligonucleotides are used in combinations. For example, a cocktail of several different LNA modified oligonucleotides is used. In some embodiments, the LNA-modified oligonucleotides are designed against different regions of the same gene. In some embodiments, the LNA-modified oligonucleotides are designed against different genes, wherein the different genes represent a family of related genes associated with a disease, a set of unrelated and diverse genes highly correlated with a disease, a set of co-regulated genes, a set of genes encoding the proteins of a biochemical or signal transduction pathway. For example, the cocktail may comprise LNA-modified oligonucleotides against the mRNAs encoding IgE and IgE-recptor (FcεRIα).

In some embodiments, the cocktail of LNA-modified oligonucleotides comprises about 2 to about 100,000 LNA-modified oligonucleotides. In some embodiments, the cocktail of LNA-modified oligonucleotides comprises more than one copy of the same LNA-modified oligonucleotide. In some embodiments, all LNA-modified oligonucleotides are unique. In some embodiments, all LNA-modified oligonucleotides in the cocktail are against one gene. In some embodiments, the LNA-modified oligonucleotides in the cocktail are against more than one gene. In some embodiments, when the LNA-modified oligonucleotides in the cocktail are against more than one gene, at least one LNA-modified oligonucleotide exists per gene. In some embodiments, the different LNA modified oligonucleotides may be administered simultaneously or separately either individually or in sets.

In some embodiments, the SMC1 LNA-modified oligonucleotide in the composition is 5′ GTATGGTTAATGGCTG 3′ (SEQ ID NO: 29). In some embodiments, the SMC1 LNA-modified oligonucleotide in the composition is 5′ ATGCCAGCCAAATTGC 3′ (SEQ ID NO: 30). In some embodiments, the cocktail of SMC1 LNA-modified oligonucleotides in the composition comprises 5′ GTATGGTTAATGGCTG 3′ (SEQ ID NO: 29) and 5′ ATGCCAGCCAAATTGC 3′ (SEQ ID NO: 30).

In some embodiments, the composition is provided as a liquid, solid or semi-solid dosage form. Non-limiting examples include capsule, tablet, an ovule, suppository, an insert, a wafer, a chewable tablet, a buccal tablet, a sub-lingual tablet, a quick-dissolve tablet, an effervescent tablet, a granule, a pellet, a bead, a pill, a sachet, sprinkle, film, ointment, a cream, a gel, a dry syrup, a reconstitutable solid, a suspension, an emulsion, a lozenge, a troche, an implant, a powder, a triturate, a platelet, or a strip. Compositions for oral administration can be any dosage form that is suitable for oral ingestion, for example, liquid compositions such as elixir, suspension, syrup, emulsion, ampoule, etc., solid compositions such as gel, gum, drop, powder, granule, pill, sugar-coated tablet, film-coated tablet, capsule, package agent, etc. Also contemplated are sustained-release compositions such as gel-coated compositions, multi-coated compositions, localized release compositions.

In some embodiments, the compositions can be formulated for immediate release, pulsatile release, controlled release, extended release, modified release, delayed release, targeted release, or targeted delayed release.

The route of administration of the compositions herein can be determined by one of ordinary skill in the art based on the circumstances and need. Several non-limiting routes of administrations are possible including parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

In some embodiments, the compositions provided herein comprise active ingredients, inactive ingredients, excipients, additives, and/or pharmaceutically acceptable carriers. Examples of additives include natural polymer compounds, inorganic salts, binders, lubricants, disintegrants, surfactants, thickeners, coating agents, pH adjusters, antioxidants, flavoring agents, preservatives, and colorants among others. Examples of other pharmaceutically acceptable carriers include liquid carriers such as water, alcohol, emulsion, and solid carriers such as gel, powder, etc. A pharmaceutically acceptable carrier may include one or more solvents, buffers, solutions, dispersion media, coatings, antibacterial and antifungal agents, isotonic and absorption delaying agents and the like acceptable for use in formulating pharmaceuticals, such as compositions suitable for administration to humans. Standard pharmaceutical formulation techniques and ingredients can be used, such as those disclosed in Remington's The Science and Practice of Pharmacy, 21st Ed., Lippincott Williams & Wilkins (2005), which is hereby incorporated by reference in its entirety. The compositions may comprise appropriate salts and buffers to render deliver vehicles stable and allow for uptake by target cells. Aqueous compositions of the present invention comprise an effective amount of the delivery vehicle comprising the LNA-modified oligonucleotide (e.g. liposomes, nanoparticles, or other complexes), dissolved or dispersed in a pharmaceutically acceptable carrier or aqueous medium. Other excipients include water soluble polymer, water insoluble polymers, hydrophobic materials, hydrophilic materials, waxes, disintegrants, superdisintegrants, diluents, binders, etc.

In some embodiments, compositions for intravenous administration comprise excipient and pharmaceutically acceptable carries including one or more of sodium chloride, dextrose, and sterile water. Compositions can comprise aqueous isotonic sterile injection solutions, which can comprise one or more of antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some embodiments, the compositions are administered by intravenous infusion. The compositions can be presented in unit-dose or multi-dose sealed containers, such as ampules and/or vials. Injection solutions and suspensions can be prepared from sterile powders, granules, and/or tablets. In some embodiments, the compositions to be administered can be formulated as pharmaceutical formulations for delivery via one or more of the routes provided herein.

As used herein, the term “subject” or “patient” refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, etc.). In some embodiments, the subject is a mammal. In some embodiments, the subject is a human.

In some embodiments, the subject receives a sufficient daily dosage of antibody-conjugates to achieve an effective yet safe concentration in the subject. In some embodiments, the subject receives a sufficient daily dosage of LNA-modified oligonucleotide to achieve an effective yet safe concentration in the subject. In some embodiments, the subject receives a sufficient daily dosage of compositions comprising antibody-conjugates and LNA-modified oligonucleotides to achieve effective yet safe concentrations of antibody-conjugates and LNA-modified oligonucleotides in the subject. Those skilled in the art should be readily able to derive appropriate dosages and schedules of administration to suit the specific circumstance and needs of the patient.

In some embodiments, the subject is a male or a female. In some embodiments, the subject is naive and never been previously received anti-cancer treatment. In some embodiments, the patient may have initially responded to the anti-cancer treatment resulting in an initial regression of the cancer. However, the car may have become resistant to the anti-cancer treatment resulting in a relapse. In some embodiments, relapse may also occur due to discontinuation of treatment, in which case the relapsed cancer may or may not be sensitive to the anti-cancer treatment previously administered. In some embodiments, the subject may initially have been treated with a first anti-cancer treatment regimen, but may subsequently have subsequently been treated with a different anti-cancer treatment regimen due to development of resistance to the first anti-cancer agent, adverse effects of the first anti-cancer agent, etc. In some embodiments, the subject has had disease for about 1 month to about 10 years.

Frequency of administration of the compositions herein can be varied depending various parameters such as patient compliance, side effects, etc., for example, daily, weekly, biweekly, monthly, bimonthly, or as is known in the art. Compositions can be administered daily, weekly, biweekly, monthly, bimonthly, less frequently, or more frequently as desired.

Administration can be daily, or 1, 2, 3, 4, 5, 6 or more times weekly, or more or less frequently as required. Administration can be provided as a single dose or as divided doses, such that a daily dose may be given in 2, 3, 4, or more portions in a single day.

In some embodiments, the subject is administered one or more additional therapeutic agent in combination with the antibody-conjugate and LNA-modified oligonucleotides. In some embodiments, the additional therapeutic agent is an anti-cancer therapeutic agent. In some embodiments, the additional anti-cancer therapeutic agent comprises PARP inhibitors. Non-limiting examples of PARP inhibitors include Niraparib (MK-4827), Iniparib (BSI 201), Talazoparib (BMN-673), Veliparib (ABT-888), Olaparib (AZD-2281), Rucaparib (AG014699, PF-01367338), CEP 9722, E7016, BGB-290, and 3-aminobenzamide. The amount and doses of PARP inhibitors are well-known in the art. For examples, in some embodiments of the amount of PARP inhibitor administered is about 0.5 mg to about 1200 mg per day. In some embodiments, PARP inhibitor is administered at a dose of about 0.0075 mg/kg to about 20 mg/kg.

In some embodiments, the additional anti-cancer therapeutic agent comprises platinum-based anti-cancer drugs are used. Non-limiting examples platinum-based anti-cancer drugs Cisplatin, Carboplatin, Oxaliplatin, Nedaplatin, Triplatin tetranitrate, Phenanthriplatin, Picoplatin, and Satraplatin. The amount and doses of platinum-based drugs are well-known in the art. In some embodiments, the subject is administered one or more additional therapeutic agents that are well-known in the art (e.g., other anti-cancer agents, anti-inflammatory drugs, etc.). In some embodiments, the additional therapeutic agents are well-known in the art and in some embodiments are approved for therapeutic use and/or use in clinical trials by government agencies (e.g., FDA, EMEA, etc.). The dosing, route of administration, efficacy against known cancer types, side/adverse effects, mechanism of action, etc. may also be well-known in the art. In other embodiments, the additional therapeutic agents are compounds that are believed to have anti-cancer effects (e.g., without being limiting, in vitro, in vivo and/or ex vivo in a laboratory and/or in a human clinical trial), but is not yet approved by a government agency for the treatment of cancer.

Co-administration of the other therapeutic agents may comprise administering the other therapeutic agents simultaneously, or within about 1, 5, 15, 30, 45 or 60 minute of one another, or within any range defined by the aforementioned values. Co-administration may comprise administering the composition and the other therapeutic agents within about 1 hour to within about 6 hours of one another, or within a range defined by any two of the aforementioned values.

In some embodiments, when the subject is administered a combination of the antibody-conjugate and LNA-modified oligonucleotides an additive effect is observed. In some embodiments, when the subject is administered a combination of the antibody-conjugate and LNA-modified oligonucleotides a synergistic effect is observed. When additionally combined with one or more additional therapeutic agents, the effect of the combination of the antibody-conjugate and LNA-modified oligonucleotides is potentiated further. In some embodiments, further potentiation is additive. In some embodiments, further potentiation is synergistic.

An additive effect is observed when the effect of a combination is equal to the sum of the effects of the individual (e.g., the effect of the combination the antibody-conjugate and the LNA-modified oligonucleotide is equal to the sum of effects of the antibody-conjugate and the LNA-modified oligonucleotide individually). A synergistic effect is greater than an additive effect. A synergistic effect is observed when the effect of a combination is equal to the sum of the effects of the individual (e.g., the effect of the combination the antibody-conjugate and the LNA-modified oligonucleotide is greater than the sum to the effects of the antibody-conjugate and the LNA-modified oligonucleotide individually). Additive effect, synergistic effect, or both can be occur human patients, non-human patients, non-patient human volunteers, in vivo models, ex vivo models, in vitro models, etc.

Synergistic effect can range from about >1 to about 100-fold. In some embodiments, the synergistic effect is about 2 to about 20-fold. In some embodiments, the synergistic effect is about 20 to about 100 fold. In some embodiments, the synergistic effect is from >1, 2, 3, 4, 5, 6, 7, 8, 9, 10, 20, 30, 40, 50, 60, 70, 80, 90, or 100-fold, or within a range defined by any two of the aforementioned values.

### Kits and Diagnostics

Any of the compositions disclosed herein can be provided as one or more of a diagnostic, prevention and/or treatment kit. In some embodiments, one or more kits are for diagnosis of one or more cancers. In some embodiments, one or more kits are for prevention of one or more cancers. In some embodiments, one or more kits are for treatment of one or more cancers. In some embodiments, one or more kits are for diagnosis, prevention and/or treatment of one or more cancers. In some embodiments, the one or more cancers are related to overexpression of SMC1.

It is within the scope of the present disclosure to provide the components of the kit such that the components of the combinations disclosed herein are suitable for co-administration.

A kit comprising one or more antibody-conjugates to be used in combination with one or more LNA-modified oligonucleotides are provided. The components of the compositions in the kit can be separately provided such as in separate containers, or in separate compartments of a divided bottle or divided foil packet (e.g., a blister pack used for the packaging of tablets, capsules, etc.). In some embodiments, the kit is suitable for administering different dosage forms, for example, oral and intravenous, for administering the components at different dosage intervals, and/or for titration of components against one another. The kit typically comprises directions for administration and may additionally be provided with a memory aid to ensure compliance.

In some embodiments, the components of the compositions in the kit may be provided in dissolved form, undissolved form or a combination thereof. For example, the antibody-conjugates may be in dissolved form and the LNA-modified oligonucleotides may be in undissolved form or vice versa. If present in undissolved form, the undissolved component may be combined with another component present in a dissolved form in a specific stoichiometric amount prior to use. If all the components are present in an undissolved form, the components can either be administered as such (e.g., orally) or dissolved into a solvent (e.g., water) prior to administration (e.g., intravenously).

Any of the embodiments of the antibody-conjugates and LNA-modified oligonucleotides provided herein can be used in methods for diagnosis. In some embodiments, the methods for diagnosis comprise, without limitation, assessment of a state of a sample, assessment of the presence of disease, etc. In some embodiments, sample is a cell suspension, tissue, biopsy, blood, urine, plasma, lymph, saliva, sputum, cerebrospinal fluid, etc. In some embodiments, the methods for diagnosing a state of a sample use the embodiments of the antibody-conjugates provided herein. In some embodiments, the methods for diagnosing a state of a sample use the embodiments of the LNA-modified oligonucleotides provided herein. In some embodiments, the methods for diagnosing a state of a sample use the embodiments of the antibody-conjugates and the embodiments of the LNA-modified oligonucleotides provided herein.

In some embodiments, an assessment of the state of a sample for diagnosis is performed using methods well known in the art such as immunohistochemical staining, flowcytometry, immunofluorescence, etc. For example, in some embodiments, an assessment is performed to determine a cellular localization of SMC1 in the sample. A localization of SMC1 only in the nucleus is indicative of a normal state of the sample. In contrast, a localization of SMC1 in the nucleus, the cytoplasmic and the plasma membrane is indicative of an abnormal state of the sample. In some embodiments, an abnormal state of the sample is indicative of the presence of a tumor. In some embodiments, an abnormal state of the sample is indicative of the presence of a cancer. In some embodiments, an abnormal state of the sample is indicative of the presence of a tumor, a cancer or a combination thereof. In some embodiments, an abnormal state of the sample is indicative of the presence of other disease states. In some embodiments, the delivery of the compositions can be used both in vitro and in vivo to perform in vitro diagnosis as well as in vivo diagnosis. For example, in some embodiments, composition administered in vivo can comprise an antibody-conjugate that is conjugated to a radionuclide which enables in vivo diagnosis using techniques and methods known in the art (e.g., CT scan, PET scan, etc).

In some embodiments, modulation of expression of a gene by one or more LNA-modified oligonucleotides (e.g., by RNA degradation and silencing) can be determined can be determined by assays well-known in the art. Non-limiting examples include RT-PCR, qPCR, real time PCR, in situ hybridization, fluorescence in situ hybridization, etc.

The following Examples are non-limiting and other variants contemplated by one of ordinary skill in the art are included within the scope of this disclosure.

### EXAMPLES

### Example 1—Generation of Antibodies Against SMC1 Epitopes

SMC1 is purified using a dinitrophenyl-S glutathione (DNP-SG) affinity resin. Mice, rats, and/or rabbits are inoculated with purified SMC1 (either as a liposomal preparation or as protein) to generate polyclonal and/or monoclonal antibodies against SMC1 epitopes. Mice, rats, and/or rabbits are inoculated peptide fragments of SMC1 (either synthesized or generated by enzymatic degradation of SMC1) to generate polyclonal and/or monoclonal antibodies against SMC1 epitopes. Non-limiting examples SMC1 epitopes are listed in Table 1.

Example 2—Localization of SMC1 in Lung Cancer Cells

Approximately 50,000 cells of the lung cancer cell line Am1010, described in Li et al., Acta Pharmacol. Sin., Vol. 31, No. 5, pp. 601-608, 2010, which is hereby incorporated by reference in its entirety, were incubated on sterile cover slips overnight with an anti-SMC1 monoclonal antibody against an epitope (SEQ ID NO: 11) of SMC1 was added at a dilution of 1:100 for 4 hours under standard tissue culture conditions. Standard tissue culture buffer was used but the buffer did not contain any reagent(s) to permeabilize the cells (typically a detergent (e.g, Triton X 100) would be added to the standard tissue culture buffer to permaebilize cells). Thereafter, cells were washed 3 times with sterile PBS. Cells were then blocked with 1:10 goat serum for 1 hour, and anti-mouse FITC-conjugated antibody at 1:500 for 1 hour. Cells were then washed 3 times with PBS, and imaged by fluorescence light microscopy. No signal was observed in “no antibody” controls. In contrast, a strong signal defining the entire cell membrane was observed with the anti-SMC1 monoclonal antibody. These data suggested cell surface expression of SMC1 in unpermeabalized cells of the lung cancer cell line Am1010. Staining experiments were also performed with permeabilized cells of the lung cancer cell line Am1010, which showed cell surface as well as nuclear staining with the anti-SMC1 monoclonal antibody against the epitope of SEQ ID NO: 11.

### Example 3—Localization of SMC1 in Breast Cancer Cells

The localization of SMC1 is determined by preparing and investigating about 75 specimens of patients with triple negative breast cancer. Immunohistochemical staining is performed using commercially available antibodies as well as antibodies disclosed herein, staining the whole length of the protein and the activation moiety, respectively. The immunohistochemical slides are interpreted by two independent pathologists with expertise in breast cancer pathology. The results included a high degree of concordance among the interpreting pathologists. Cytoplasmic and membranous presence of the SMC1 protein is observed in all triple negative breast cancer cells. In contrast, SMC1 is strictly localized in the nucleus in the surrounding healthy stromal and epithelial cells. Thus, SMC1 is localized in the cytoplasm and on the membrane of malignant breast cells but not benign breast cells. FACS analysis is also used to confirm the surface localization of SMC1.

### Example 4—Localization of SMC1 in Glioblastoma Cells

The localization of SMC1 in glioblastoma cells is determined by preparing and investigating about 75 specimens from patients with glioblastoma multiforme. Immunohistochemical staining is performed, using an anti-SMC1 polyclonal antibody that binds to epitopes along the whole length of SMC1. The immunohistochemical staining slides are interpreted by two independent pathologists with expertise in brain cancer pathology. Cytoplasmic and membranous presence of the SMC1 protein is observed in all glioblastoma cells. In contrast, SMC1 is strictly localized in the nucleus in the surrounding healthy stromal and epithelial cells. Thus, SMC1 is localized in the cytoplasm and on the membrane of glioblastoma cells. The results are highly concordant among the interpreting pathologists. FACS analysis is also used to confirm the surface localization of SMC1.

### Example 5—Selective Cytotoxicity Against Malignant Cells

SMC1 is a well-described structural component of the cohesion complex and intimately involved with the partition of sister chromatids in mitosis. Thus, reducing SMC1 expression in malignant cells would be more toxic to malignant cells than to healthy cells. A series of cytotoxicity experiments are conducted with brain cancer cells, lung cancer cells, ovarian cancer cells and mesothelioma cells. HUVEC cells are used as control. MTT and LDH assays are performed which are well-known in the art. The efficacy of LNA-modified oligonucleotides in reducing RNA levels is determined by assays that are well-known in the art (e.g., RT-PCR, luciferase assay, etc.). Targeting SMC1 using anti-SMC1 monoclonal antibody-conjugate and SMC1 LNA-modified oligonucleotides leads to cell death in the malignant cell populations, while healthy, normal cells are unharmed. anti-SMC1 monoclonal antibody-conjugate and SMC1 LNA-modified oligonucleotides show selective cytotoxicity against malignant cells, while leaving healthy cells unharmed.

### Example 6—Synergism with PARP Inhibitors and Cisplatin

Based on the hypothesis that reducing SMC1 in malignant cells would potentiate the cytotoxicity of cisplatin and PARP inhibitors, a series of cytotoxicity experiments are conducted with breast cancer cells, brain cancer cells, lung cancer cells, ovarian cancer cells and mesothelioma cells. The effect of a combination of anti-SMC1 monoclonal antibody-conjugate, SMC1 LNA-modified oligonucleotides, PARP inhibitors and Cisplatin on cells are tested using MTT and LDH assays. The combination of anti-SMC1 monoclonal antibody-conjugate, SMC1 LNA-modified oligonucleotides, PARP inhibitors and Cisplatin leads to increased cell death in the malignant cell populations.

### Example 7—Preferential Killing of Cancer Stem Cell Population

It is known that cancers are maintained by cancer stem cell populations. Even though cancer stem cells initially show complete response to anti-cancer therapy, the cancers inevitably recur, usually with more aggressive features. It is hypothesized that targeting the cancer stem cell populations could prevent and/or eliminate such cancers. Therefore, a series of experiments are conducted on breast cancer cells, brain cancer cells, lung cancer cells, and ovarian cancer cells by isolating their stem cell population and exposing the stem cell populations to a combination of anti-SMC1 monoclonal antibody-conjugate and SMC1 LNA-modified oligonucleotides. Increased cell death in malignant cancer stem cell populations is observed with the combination of anti-SMC1 monoclonal antibody-conjugate and SMC1 LNA-modified oligonucleotides. Thus, cancer stem cell populations are preferentially targeted and killed by anti-SMC1 monoclonal antibody-conjugate and SMC1 LNA-modified oligonucleotides.

### Example 8—SMC1 is Overexpressed in Cancer Cell Lines

The expression of SMC1 at the RNA and protein levels is assessed in cancer cell lines. A non-tumorigenic cell line is used as control. The mRNA expression of SMC1, assessed by quantitative RT-PCR, is higher in cancer cell lines by about 1.5-fold to about 15-fold as compared to the non-tumorigenic cell line. Expression of SMC1 protein is assessed by Western blotting using anti-SMC1 antibodies disclosed herein. Densitometry analysis of Western blots shows about 1.5-fold to about 15-fold increase in SMC1 protein expression in the cancer cell lines as compared to the non-tumorigenic cell line.

### Example 9—Synergistic Effect of SMC1 LNA-Modified Oligonucleotides and PARP Inhibitors Against Cancer Cell Lines

The effect on cancer cell lines of a combination of suppression of SMC1 mRNA expression (by known RNA degradation and silencing mechanisms) using LNA-modified oligonucleotides disclosed herein and one or more PARP inhibitors is assessed. The effect of LNA-modified oligonucleotides alone and PARP inhibitors alone is also assessed using known standard cell growth assays. HUVEC cell are used as control. The combination of LNA-modified oligonucleotides and PARP inhibitors results in sensitization of cancer cell lines to PARP inhibitors such that the IC₅₀ of the PARP inhibitors against the cell lines tested is lowered by about 5-fold to about 50-fold.

### Example 10—Effect of SMC1 Expression on Cell Migration

The effect of modulating expression of SMC1 on cell migration of cancer cell lines is assessed. Cancer cell lines overexpressing SMC1 migrate efficiently. Cancer cell lines overexpressing SMC1 are transfected with SMC1 LNA-modified oligonucleotides in liposomes to suppress expression of SMC1. Suppression of SMC1 expression results in less efficient migration of cancer cell lines as compared to untransfected cancer cells. The migration efficiency is decreased by about 1.5-fold to about 15-fold.

### Example 11—Effect of SMC1 Expression on Vimentin and E-Cadherin

Effect of suppression of SMC1 expression on vimentin and E-cadherin in cancer cell lines is assessed. Vimentin regulates epithelial-mesenchymal transition and expression of vimentin is associated with enhanced motility and metastasis of tumor cells. E-cadherin is required to maintain the epithelial phenotype of the basement membrane. Cell lines overexpressing SMC1 are transfected with SMC1 LNA-modified oligonucleotides in liposomes to suppress expression of SMC1. Suppression of SMC1 expression results in suppression of vimentin expression and enhancement of E-cadherin expression by about 1.5-fold to about 150-fold.

### Example 12—Effect of SMC1 on Colony Formation

Effect of modulating SMC1 expression on colony forming activity of tumorigenic cell lines is examined. Cell lines overexpressing SMC1 are transfected with SMC1 LNA-modified oligonucleotides in liposomes to suppress expression of SMC1. Appropriate controls are used. Depletion of SMC1 causes decrease in colony formation and anchorage-independent growth by about 10% to about 90%. Depletion of SMC1 causes about 5% to about 75% apoptosis the tumorigenic cell lines as determined using TUNEL-assay.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entireties. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

Although this disclosure is in the context of certain embodiments and examples, those of ordinary skill in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of ordinary skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

1-46. (canceled)

47. An antibody-conjugate comprising an anti-SMC1 antibody, wherein the antibody is conjugated to a cytotoxic molecule via a linker, wherein the antibody binds to one or more epitopes in an extracellular C-terminal region of SMC1.

48. The antibody-conjugate of claim 47, characterized by one or more of the following features:
(a) wherein the antibody binds to an epitope in the extracellular C-terminal region of SMC1 comprising residues 805-1233 of SMC1;
(b) wherein the epitope is selected from the group consisting of the sequences listed in Table 1;
(c) wherein the epitope is selected from the group consisting of SEQ ID NO: 11, SEQ ID NO: 12 and SEQ ID NO: 13;
(d) wherein the antibody is monoclonal or polyclonal;
(e) the cytotoxic molecule is selected from the group consisting of calicheamicin, maytansinoids, auristatins, taxol, cytochalasin B, gramicidin D, ethidium bromide, emetine, mitomycin, vinca alkaloids, etoposide, tenoposide, vincristine, vinblastine, colchicin, doxorubicin, daunorubicin, dihydroxy anthracinedione, mitoxantrone, mithramycin, actinomycin D, methotrexate, adriamicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including and analogs, homologs, fragments and/or variants thereof, 1-dehydrotestosterone, glucocorticoids, procaine, tetracaine, lidocaine, propranolol, and puromycin, ricin, CC-1065, duocarmycins, diptheria toxin, venom (e.g., from snakes, amphibians, reptiles, fish, invertebrates, etc.), and analogs, homologs, fragments, and/or variants thereof, bismuth-213, astatine-211 , radium-223, yttrium-90, iodine-131, samarium-153, strontium-89, lutetium-177, holmium-166, rhenium-186, rhenium-188, copper-67, promethium-149, gold-199, rhodium-105, bromine-77, indium-111, iodine-123, and iodine-125; and
(f) wherein the antibody-conjugate specifically binds a cell that has surface expression of SMC1.

49. An LNA-modified oligonucleotide comprising one or more LNAs, wherein the LNA-modified oligonucleotide is complementary to an mRNA encoding SMC1, and wherein the LNA-modified oligonucleotide binds the mRNA encoding SMC1 and targets the mRNA encoding SMC1 for degradation by an RNA silencing mechanism.

50. The LNA-modified oligonucleotide of claim 49, characterized by one or more of the following features:
(a) wherein the length of the LNA-modified oligonucleotide is about 5 to about 50 nucleotides;
(b) wherein the sequence of the LNA-modified oligonucleotide is selected from the group consisting of 5′ GTATGGTTAATGGCTG 3′ (SEQ ID NO: 29) and 5′ ATGCCAGCCAAATTGC 3′ (SEQ ID NO: 30);
(c) wherein the number of LNAs in the LNA-modified oligonucleotide is about 1 to about 25; and
(d) wherein one or more of the nucleotides in SEQ ID NO: 29 and SEQ ID NO: 30 are LNAs.

51. A composition for preventing and/or treating a disease in a subject, the composition comprising:
an antibody-conjugate comprising an anti-SMC1 antibody, wherein the antibody is conjugated to a cytotoxic molecule via a linker, wherein the antibody binds to one or more epitopes in an extracellular C terminal region of SMC1; and
an LNA-modified oligonucleotide comprising one or more LNAs, wherein the LNA-modified oligonucleotide is complementary to an mRNA encoding SMC1, and wherein the LNA-modified oligonucleotide binds the mRNA encoding SMC1 and targets the mRNA encoding SMC1 for degradation by an RNA silencing mechanism.

52. The composition of claim 51, characterized by one or more of the following features:
(a) wherein the antibody binds to an epitope in the extracellular C terminal region of SMC1 comprising residues 805-1233 of SMC1;
(b) wherein the epitope is selected from the group consisting of the sequences listed in Table 1;
(c) wherein the epitope is selected from the group consisting of SEQ ID NO: 11, SEQ ID NO: 12 and SEQ ID NO: 13;
(d) wherein the antibody is monoclonal or polyclonal;
(e) the cytotoxic molecule is selected from the group consisting of calicheamicin, maytansinoids, auristatins, taxol, cytochalasin B, gramicidin D, ethidium bromide, emetine, mitomycin, vinca alkaloids, etoposide, tenoposide, vincristine, vinblastine, colchicin, doxorubicin, daunorubicin, dihydroxy anthracinedione, mitoxantrone, mithramycin, actinomycin D, methotrexate, adriamicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including and analogs, homologs, fragments and/or variants thereof, 1-dehydrotestosterone, glucocorticoids, procaine, tetracaine, lidocaine, propranolol, and puromycin, ricin, CC-1065, duocarmycins, diptheria toxin, venom (e.g., from snakes, amphibians, reptiles, fish, invertebrates, etc.), and analogs, homologs, fragments, and/or variants thereof, bismuth-213, astatine-211, radium-223, yttrium-90, iodine-131, samarium-153, strontium-89, lutetium-177, holmium-166, rhenium-186, rhenium-188, copper-67, promethium-149, gold-199, rhodium-105, bromine-77, indium-111, iodine-123, and iodine-125;
(f) wherein the antibody-conjugate specifically binds a cell that has surface expression of SMC1.
(g) wherein the length of the LNA-modified oligonucleotide is about 5 to about 50 nucleotides;
(h) wherein the sequence of the LNA-modified oligonucleotide is selected from the group consisting of 5′ GTATGGTTAATGGCTG 3′ (SEQ ID NO: 29) and 5′ ATGCCAGCCAAATTGC 3′ (SEQ ID NO: 30);
(i) wherein the number of LNAs in the LNA-modified oligonucleotide is about 1 to about 25;
(j) wherein one or more of the nucleotides in SEQ ID NO: 29 and SEQ ID NO: 30 are LNAs;
(k) wherein the preventing and/or treating is achieved by modulating an expression of SMC1 mRNA and SMC1 protein; and
(l) wherein the disease is selected from the group consisting of breast cancer (e.g., triple negative breast cancer), breast adenocarcinoma, pancreatic adenocarcinoma, lung carcinoma, prostate cancer, hormone refractory prostate cancer, solid tumor malignancies such as colon carcinoma, non-small cell cancer (e.g., non-small cell lung cancer), anaplastic astrocytoma, glioma, glioblastoma (e.g., glioblastoma multiforme), bladder carcinoma, sarcoma, ovarian cancer, rectal hemangiopericytoma, pancreatic carcinoma, acute myeloid leukemia, cancer of large bowel, mesothelioma, stomach, pancreas, ovaries, melanoma, pancreatic cancer, colon cancer, and bladder cancer.

53. A kit for preventing and/or treating a disease in a subject, the kit comprising a composition according to claim 51.

54. The kit of claim 53, wherein the disease is selected from the group consisting of breast cancer (e.g., triple negative breast cancer), breast adenocarcinoma, pancreatic adenocarcinoma, lung carcinoma, prostate cancer, hormone refractory prostate cancer, solid tumor malignancies such as colon carcinoma, non-small cell lung cancer (e.g., non-small cell lung cancer), anaplastic astrocytoma, glioma, glioblastoma (e.g., glioblastoma multiforme), bladder carcinoma, sarcoma, ovarian cancer, rectal hemangiopericytoma, pancreatic carcinoma, acute myeloid leukemia, cancer of large bowel, mesothelioma, stomach, pancreas, ovaries, melanoma, pancreatic cancer, colon cancer, and bladder cancer.

55. A method of preventing and/or treating a disease in a subject, the method comprising:
performing a first assessment of the disease in the subject;
providing the composition of claim 51;
administering the composition to the subject for a duration of time;
performing a second assessment of the disease in the subject after the duration of time, wherein the second assessment of the disease indicates prevention and/or treatment of the disease in the subject after administering the composition to the subject for a duration of time;
thereby preventing and/or treating the disease in the subject.

56. The method of claim 55, characterized by one or more of the following features:
(a) wherein the cell is a tumor cell, cancer cell, tumor stem cell, cancer stem cell, or a combination thereof;
(b) wherein the antibody-conjugate is taken up by the cell, wherein the cytotoxic molecule preferably either arrests the growth of the cell or kills the cell;
(c) wherein the LNA-modified oligonucleotide is formulated with a fusogenic or lipogenic component that allows an uptake of the LNA-modified oligonucleotide by a cell, wherein the cell preferably overexpresses an SMC1 mRNA, wherein the LNA-modified oligonucleotide preferably is complementary to the mRNA of SMC1, and wherein the LNA-modified oligonucleotide binds the SMC1 mRNA and modulates expression by an RNA silencing mechanism;
(d) wherein the subject is a mammal, wherein the mammal is a human or a non-human;
(e) further comprising providing one or more additional therapeutic agents, wherein the one or more additional therapeutic agents preferably is a PARP inhibitor, and wherein the one or more PARP inhibitor preferably is selected from the group consisting of Niraparib (MK-4827), Iniparib (BSI 201), Talazoparib (BMN-673), Veliparib (ABT-888), Olaparib (AZD-2281), Rucaparib (AG014699, PF-01367338), CEP 9722, E7016, BGB-290, and 3-aminobenzamide, wherein the one or more additional therapeutic agents preferably is a platinum-based drug preferably selected from the group consisting of Cisplatin, Carboplatin, Oxaliplatin, Nedaplatin, Triplatin tetranitrate, Phenanthriplatin, Picoplatin, and Satraplatin;
(f) wherein the one or more additional therapeutic agents potentiates the effect of the composition;
(g) wherein the concertation of the antibody-conjugate in the composition ranges from about 1 nM to about 250 mM; and
(h) wherein the concentration of the LNA-modified oligonucleotide in the composition ranges from about 1 nM to about 250 mM.

57. A method of diagnosing a state of a sample, the method comprising:
providing a sample wherein the sample is a cell suspension, tissue, biopsy or a combination thereof;
providing an anti-SMC antibody;
performing an immunohistochemical staining of the sample using the SMC1 antibody;
determining a cellular localization of SMC1 in the sample, wherein a localization of SMC1 only in the nucleus is indicative of a normal state of the sample, and wherein a localization of SMC1 in the nucleus, the cytoplasmic and the plasma membrane is indicative of an abnormal state of the sample.

58. The method of claim 57, characterized by or both of the following features:
(a) wherein the abnormal state of the sample is indicative of the presence of a disease; and
(b) wherein the disease is selected from the group consisting of breast cancer (e.g., triple negative breast cancer), breast adenocarcinoma, pancreatic adenocarcinoma, lung carcinoma, prostate cancer, hormone refractory prostate cancer, solid tumor malignancies such as colon carcinoma, non-small cell cancer (e.g., non-small cell lung cancer), anaplastic astrocytoma, glioma, glioblastoma (e.g., glioblastoma multiforme), bladder carcinoma, sarcoma, ovarian cancer, rectal hemangiopericytoma, pancreatic carcinoma, acute myeloid leukemia, cancer of large bowel, mesothelioma, stomach, pancreas, ovaries, melanoma, pancreatic cancer, colon cancer, and bladder cancer.
